(19) United States
(12) Patent Application Publication
(10) Pub. No.: US 2022/0387304 A1
(43) Pub. Date: Dec. 8, 2022

(54) MULTI-LAYERED NANOPARTICLE COATED SUBSTRATES FOR DRUG DELIVERY

(71) Applicant: The UAB Research Foundation, Birmingham, AL (US)

(72) Inventors: Do-Yeon CHO, Birmingham, AL (US); Dong Jin LIM, Birmingham, AL (US); Bradford A. WOODWORTH, Mountainbrook, AL (US)

(21) Appl. No.: 17/765,926

(22) PCT Filed: Oct. 2, 2020
(86) PCT No.: PCT/US2020/053986

§ 371 Date: Apr. 1, 2022

Related U.S. Application Data
(60) Provisional application No. 62/909,840, filed on Oct. 3, 2019.

(51) Int. Cl. A61K 9/00; A61K 31/496; A61K 31/7052; A61K 31/47; A61L 31/10; A61L 31/16

(52) U.S. Cl. CPC A61K 9/0024; A61K 31/496; A61K 31/7052; A61K 31/47; A61L 31/10; A61L 31/16; A61L 2300/406; A61L 2300/624; A61L 2400/12

(57) ABSTRACT

Disclosed herein are bilayered substrates useful for treating infection and/or inflammation in a subject such as, for example, the upper respiratory system. In another aspect, the layers of the substrates disclosed herein include biocompatible and biodegradable polymers as well as one or more bioactive agents useful for treating infection and/or inflammation. In a further aspect, the layers of the substrate can contain nanoparticles incorporating the bioactive agents. In any one of the above aspects, the bioactive agents are released at a constant rate over a period of time. In still another aspect, the substrates disclosed herein are useful for reducing the mass of biofilms and reducing or preventing inflammation by inhibiting the production of interleukin-8.

### CROSS REFERENCE TO RELATED APPLICATIONS

This application claims priority upon U.S. provisional application Ser. No. 62/909,840 filed on Oct. 3, 2019. This application is hereby incorporated by reference in its entirety.

### BACKGROUND

Chronic rhinosinusitis (CRS) is a chronic inflammatory and infectious process of the sinus and nasal cavities and is a common chronic disease, afflicting 14-16% of the adult population of the United States. Bacterial biofilms, or aggregates of extracellular polysaccharides, are likely a key modulator of the refractory nature of CRS and increase the tolerance of bacteria to antibiotics through numerous mechanisms. Bacterial biofilms have been found on the sinonasal mucosa of up to 54% of CRS sufferers, compared to 8% of control patients. Multiple studies have noted a higher prevalence of biofilms in patients who are undergoing revision sinus surgery. In particular, the presence of biofilm-forming Pseudomonas aeruginosa strains has been associated with poor resolution of symptoms and signs of CRS following endoscopic sinus surgery. Bacterial biofilms produced by certain pathogens like P. aeruginosa reduce antibiotic penetration and lead to interventional failure in recalcitrant CRS.

Sufficient antibiotic exposure is needed to ensure the eradication of the microorganisms; thus, antibiotic treatments often involve a long course of therapy. To avoid systemic side effects, topical drug-eluting implants with prolonged mucosal contact time and sustained drug release may provide a suitable therapeutic option. This approach should allow for a larger dose and more efficient localized delivery of the drug to penetrate into biofilms, resulting in a potent therapeutic effect while avoiding adverse systemic side effects associated with long-term systemic antibiotic treatment. In addition, agents that enhance the antimicrobial activity of currently available antibiotics may further represent a valuable and cost-effective means for improving clinical efficacy.

What is needed is a local method of treatment of CRS with antibiotics and/or other agents that enables avoidance of systemic side effects associated with traditional antibiotic treatment. Ideally, this method would release drugs over time in a controlled, sustained manner, with no initial burst. The method would further combine multiple drugs into one delivery system, offering synergistic effects that include reducing or eliminating of biofilms, reducing the likelihood of developing antibiotic resistance, and providing additional benefits such as anti-inflammatory effects or enhancement of mucociliary clearance, thus improving symptoms while also treating infections. Ideally, this method would employ a biocompatible, biodegradable, non-toxic delivery device. The present disclosure addresses these and other needs.

### SUMMARY

In one aspect, disclosed herein are bilayered substrates useful for treating infection and/or inflammation in a subject such as, for example, the upper respiratory system. In another aspect, the layers of the substrates disclosed herein include biocompatible and biodegradable polymers as well as one or more bioactive agents useful for treating infection and/or inflammation. In a further aspect, the layers of the substrate can contain nanoparticles incorporating the bioactive agents. In any one of the above aspects, the bioactive agents are released at a constant rate over a period of time. In still another aspect, the substrates disclosed herein are useful for reducing the mass of biofilms and reducing or preventing inflammation by inhibiting the production of interleukin-8.

The advantages of the materials, methods, and devices described herein will be set forth in part in the description that follows, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIGS. 1A-1C show scanning electron microscopy (SEM) images of (A) ciprofloxacin-loaded nanoparticles, (B) ivacaftor-loaded nanoparticles, and (C) a mixture of ciprofloxacin and ivacaftor nanoparticles (scale bar 10 μm).

FIGS. 2A-2C show SEM images of a ciprofloxacin- and ivacaftor-releasing biodegradable sinus stent (CISS). (A) shows a top-down view of the CISS surface; (B) shows a cross-sectional view of the dual layers of CISS before use; and (C) shows a cross-sectional view of the dual layers of CISS 21 days after in vitro release. An asterisk (*) indicates the inner layer of the stent; double asterisks (**) indicate the outer layer of the stent. The double-headed arrows in panels (B) and (C) show the thickness of each respective layer.

FIG. 3 shows an in vitro release profile of the CISS over a 21-day period.

FIG. 4 shows the effect of CISS on Pseudomonas aeruginosa strain PAO-1 biofilms; CISS significantly reduced biofilm mass for n=3 samples. *, **, and *** indicate statistical significance when compared to a control (<0.05, <0.01, and <0.0001, respectively).

FIGS. 5A-5C show the effect of the CISS on biofilm formation. (A) and (B) are representative confocal laser scanning microscopy (CLSM) of PAO-1 biofilms with (panel B) and without (panel A) CISS after 24 hours. Orthogonal images of Z-stacks show a plane view (square) looking down the biofilm and side views through the biofilm (to the right of and below panels A and B). The scale bar indicates 20 μm. (C) shows the percentage of live cells for a control and for CISS after 24 hours.

FIGS. 6A-6C show efficacy of CISS against preformed PAO-1 biofilms. Representative CLSM images of PAO-1 biofilms grown for three days with (panel B) or without (panel A) CISS on the preformed biofilms. Orthogonal images of Z-stacks show a plane view (square) looking down the biofilm and side views through the biofilm (to the right of and below panels A and B). The scale bar indicates 20 μm. (C) shows the percentage of live cells for a control and for CISS.

FIGS. 7A and 7B show SEM images of a ciprofloxacin and azithromycin sinus stent (CASS). An asterisk (*) indicates the ciprofloxacin (inner) layer, while a double asterisk (**) indicates the azithromycin (outer) layer. (A) shows a cross-sectional view of a single layer of ciprofloxacin. (B) shows a cross-sectional view of the dual layers of ciprofloxacin and azithromycin.

FIGS. 8A and 8B show in vitro release profiles of ciprofloxacin and azithromycin for 28 days. (A) shows ciprofloxacin-releasing profiles from single-coated ciprofloxacin stents (open circles, n=3) and dual-coated ciprofloxacin-azithromycin stents (closed squares, n=3). (B) shows an azithromycin-releasing profile from the CASS (closed circles, n=3).

FIG. 9 shows the effect of CASS on the inhibition of PAO-1 biofilm formation. CASS significantly reduced biofilm mass for n=3 samples. *** and **** indicate statistical significance when compared to a control (<0.001 and <0.0001, respectively).

FIG. 10 shows the effect of CASS on preformed PAO-1 biofilms after 1 day. CASS significantly reduced the final PAO-1 biofilm mass for n=3 samples. * and ** indicate statistical significance when compared to a control (<0.05 and <0.01, respectively).

FIGS. 11A-11C show the effect of CASS on PAO-1 biofilm formation. CLSM images of PAO-1 biofilms are shown with (panel B) and without (panel A) CASS after 24 hours. The maximum intensity projection images were used to create each panel. A plane view (square) shows the biofilm while the right and bottom images of panels (A) and (B) display the side view of the biofilm. The scale bar indicates 50 μm. Panel (C) shows the percentage of live cells in a control and in the CASS treatment.

FIGS. 12A-12C show the efficacy of CASS against preformed PAO-1 biofilms. Representative CLSM images of PAO-1 biofilms were captured after a 3-day cultivation period with (panel B) or without (panel A) placing CASS. A plane view (square) shows the biofilm while the right and bottom images of panels (A) and (B) display the side view of the biofilm. The scale bar indicates 50 μm. Panel (C) shows the percentage of live cells in a control and in the CASS treatment.

FIG. 13 shows the comparison of LPS-induced Interleukin-8 (IL-8) levels at varying concentrations of azithromycin. Reduced interleukin-8 expression by different azithromycin concentrations from P. aeruginosa lipopolysaccharide (LPS)-treated HSNEC (n=3). *, **, *** and ****: p<0.05, p<0.01, p<0.001, and p<0.0001, respectively. HSNEC: Human sinonasal epithelial cell

FIG. 14 shows the comparison of LPS-induced Interleukin-8 (IL-8) levels in the presence or absence of the study drugs. Reduced interleukin-8 expression by a combination of azithromycin and ciprofloxacin concentrations from P. aeruginosa lipopolysaccharide (LPS)-treated HSNEC (n=3). **, *** and ****: p<0.01, p<0.001, and p<0.0001, respectively. HSNEC: Human sinonasal epithelial cells

FIG. 15 shows the normalized transepithelial electrical resistance (TEER) in the presence of the study drugs. No significant reduction in normalized TEER over time in the presence of azithromycin (30 μg/ml) or azithromycin/ciprofloxacin (30 μg/ml and 0.5 μg/ml, respectively). TEER: Transepithelial electrical resistance

FIG. 16 shows the paracellular permeability in the presence and absence of the study drugs. No alterations in average paracellular permeability of HSNECs over time in the presence of azithromycin and/or ciprofloxacin. Each value is the mean+/−standard deviation of 4 samples at each time point. HSNEC: Human sinonasal epithelial cell

FIG. 17 shows the Ciliary Beat Frequency (CBF) in the presence and absence of the study drugs. No significant difference in ciliary beat frequency (CBF) was measured in the presence of azithromycin and/or ciprofloxacin. The CBF fold changes were described as mean+/−standard deviation of 4 samples at each time point.

FIG. 18 shows the lactate dehydrogenase (LDH) levels in the presence and absence of the study drugs. No detrimental effect on the cellular viability of HSNECs over time in the presence of azithromycin and/or ciprofloxacin compared to controls. The LDH values are presented as mean+/−standard deviation of 4 samples at each time point. HSNEC: Human sinonasal epithelial cell

### DETAILED DESCRIPTION

Before the present materials, articles, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In the specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “an antibiotic” includes mixtures of two or more such antibiotics, and the like.

“Optional” or “optionally” means that the subsequently described event or circumstance can or cannot occur, and that the description includes instances where the event or circumstance occurs and instances where it does not.

As used herein, the term “about” is used to provide flexibility to a numerical range endpoint by providing that a given value may be “a little above” or “a little below” the endpoint without affecting the desired result. For purposes of the present disclosure, “about” refers to a range extending from 10% below the numerical value to 10% above the numerical value. For example, if the numerical value is 10, “about 10” means between 9 and 11 inclusive of the endpoints 9 and 11.

Throughout this specification, unless the context dictates otherwise, the word “comprise,” or variations such as “comprises” or “comprising,” will be understood to imply the inclusion of a stated integer or step or group of integers or steps, but not the exclusion of any other integer or step or group of integers or steps. It is also contemplated that the term “comprises” and variations thereof can be replaced with other transitional phrases such as “consisting of” and “consisting essentially of.”

“Admixing” or “admixture” refers to a combination of two components together when there is no chemical reaction or physical interaction. The terms “admixing” and “admixture” can also include the chemical interaction or physical interaction among any of the components described herein upon mixing to produce the composition. The components can be admixed alone, in water, in another solvent, or in a combination of solvents.

The term “subject” as defined herein is any organism in need of treatment and/or prevention (e.g., infection, inflammation, etc.). In one aspect, the subject is a mammal including, but not limited to, humans, domesticated animals (e.g., dogs, cats, horses), livestock (e.g., cows, pigs), and wild animals.

The term “treat” as used herein is defined as maintaining or reducing the symptoms of a pre-existing condition in a subject when compared to the same subject that has not been administered a substrate as described herein. For example, the compositions described herein can be used to treat an infection or inflammation.

The term “prevent” as used herein is defined as eliminating or reducing the likelihood of occurrence of one or more symptoms of a disease or disorder in a subject when compared to the same subject that has not been administered a substrate as described herein. For example, the compositions described herein can be used to prevent the growth of bacteria or the onset of infection or inflammation.

The term “inhibit” as used herein is the ability of the substrates described herein to completely eliminate an activity or reduce the activity when compared to the same activity in the absence of the substrate. For example, the substrates described herein can be used to inhibit the formation of biofilms.

“Chronic rhinosinusitis” or CRS is a condition involving inflammation of the nasal and sinus mucosa. CRS can last for eight weeks or more. In some aspects, CRS is caused by bacteria, including bacteria that form biofilms in and/or on the nasal and sinus mucosa, and can be characterized by drainage, nasal congestion, difficulty breathing through the nose, and pain and/or inflammation around and/or behind the eyes. In one aspect, provided herein are devices, compositions, and methods for treating or preventing chronic rhinosinusitis.

As used herein, “bioactive agent” refers to any chemical compound or composition that has a therapeutic effect on a subject. In a further aspect, a bioactive agent can be an antibacterial compound or can provide relief from a symptom such as, for example, by improving mucociliary clearance. In one aspect, the devices, compositions, and methods disclosed herein incorporate one or more bioactive agents.

An “antibiotic” is a chemical compound or composition that treats and/or prevents bacterial infections by killing or preventing the reproduction of bacteria. In some aspects, the compositions disclosed herein are formulated to incorporate an antibiotic. An antibiotic can be naturally secreted by a microorganism as a defense compound against other microorganisms, can be a semisynthetically modified variant thereof, or can be wholly synthesized in a laboratory.

As used herein, the term “bactericidal” refers to an article, compound, or composition that kills bacteria. In one aspect, a bactericidal compound or composition can include an antibiotic.

As used herein, “burst release” refers to an initial, high-level release of a bioactive agent from an implanted device or polymeric composition that incorporates the bioactive agent. In a further aspect, the compositions disclosed herein are formulated to avoid a burst release and instead to release compounds gradually over a predetermined treatment period.

As used herein, a “biofilm” is a collection of microorganisms that stick to one another and also to a surface. In one aspect, the cells are embedded within an extracellular polymeric matrix that is produced by the cells and that typically contains polysaccharides, proteins, lipids, and occasionally nucleic acids. Biofilms can have three-dimensional structure and can form on both living and non-living surfaces. In one aspect, being embedded in a biofilm can offer some protection from antibiotics to a microorganism. In another aspect, the devices and methods disclosed herein are useful in combating existing biofilms as well as inhibiting the formation of new biofilms.

A “nanoparticle” as used herein is a particle with a size between about 1 nm and about 1000 nm. In some aspects, a nanoparticle can have an interfacial layer containing ions or molecules, which can, in turn, modulate its properties. In one aspect, the methods disclosed herein make use of nanoparticles formed from biocompatible and biodegradable polymers. In another aspect, the nanoparticles can encapsulate a bioactive agent or may have a bioactive agent in or on their surface interfacial layers.

As used herein, “zeta potential” refers to electrokinetic potential in colloids and has units of volts (V) or millivolts (mV). In some aspects, zeta potential is an indicator of stability of the colloid, with its magnitude being representative of the degree of electrostatic repulsion between adjacent, similarly-charged particles. For small particles, a high zeta potential can be an indication of stability and/or resistance to aggregation. Zeta potential can be positive or negative.

“Biodegradable” materials are capable of being decomposed by bacteria, fungi, or other organisms, or by enzymes in the body of a subject.

“Biocompatible” materials are materials that perform their desired functions without eliciting harmful or deleterious changes to the subject in which they are implanted or to which they are applied, either locally or systemically. In one aspect, the polymers disclosed herein are biocompatible.

References in the specification and concluding claims to parts by weight, of a particular element in a composition or article, denote the weight relationship between the element or component and any other elements or components in the composition or article for which a part by weight is expressed. Thus, in a compound containing 2 parts by weight of component X and 5 parts by weight of component Y, X and Y are present at a weight ratio of 2:5, and are present in such a ratio regardless of whether additional components are contained in the compound. A weight percent of a component, unless specifically stated to the contrary, is based on the total weight of the formulation or composition in which the component is included.

As used herein, a plurality of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an illustration, a numerical range of “about 1 to about 5” should be interpreted to include not only the explicitly recited values of about 1 to about 5, but also to include individual values and sub-ranges within the indicated range. Thus, included in this numerical range are individual values such as 2, 3, and 4, sub-ranges such as from 1-3, from 2-4, from 3-5, etc., as well as 1, 2, 3, 4, and 5, individually. The same principle applies to ranges reciting only one numerical value as a minimum or a maximum. Furthermore, such an interpretation should apply regardless of the breadth of the range or the characteristics being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference to each various individual and collective combination and permutation of these compounds may not be explicitly disclosed, each is specifically contemplated and described herein. For example, if an antibiotic agent is disclosed and discussed and a number of different biocompatible polymers are discussed, each and every combination of antibiotic agent and biocompatible polymer that is possible is specifically contemplated unless specifically indicated to the contrary. For example, if a class of molecules A, B, and C are disclosed, as well as a class of molecules D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered from disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such combination is specifically contemplated and should be considered disclosed.

### Coated Substrates

Described herein are coated substrates having multiple layers. In one aspect, described herein is a substrate comprising a first surface, wherein a first layer (also referred to herein as “the inner layer”) comprising a first bioactive agent is adjacent to the first surface of the substrate, and a second layer (also referred to herein as “the outer layer”) comprising a second bioactive agent is adjacent to the first layer, wherein the second bioactive agent is more hydrophobic than the first bioactive agent. In certain aspects, the first layer can include one or more inner layers composed of the same or different material. In other aspects, the second or outer layer includes a single layer, where a surface of the second or outer layer is exposed and can come into contact with tissue of a subject when administered to the subject.

Each layer can include one or more bioactive agents having varying physical and biological properties. In one aspect, the coated substrate includes a first layer having a first bioactive agent adjacent to the surface of the substrate, and a second layer having a second bioactive agent adjacent to the first layer. In some aspects, the second bioactive agent is more hydrophobic than the first bioactive agent. In one aspect, the second bioactive agent is from about 20% to about 80% more hydrophobic than the first bioactive agent, or is from about 40% to about 60% more hydrophobic than the first bioactive agent, or is about 20, 25, 30, 35, 40, 45, 50, 55, 60, 65, 70, 75, or about 80% more hydrophobic than the first bioactive agent, or a combination of any of the foregoing values, or a range encompassing any of the foregoing values. In another aspect, the second bioactive agent is completely insoluble in water. In another aspect, the second bioactive agent is from about 20% to about 80% less soluble in water than the first bioactive agent, or is from about 40% to about 60% less soluble in water than the first bioactive agent, or is about 20, 25, 30, 35, 40, 45, 50, 55, 60, 65, 70, 75, or about 80% less soluble in water than the first bioactive agent, or a combination of any of the foregoing values, or a range encompassing any of the foregoing values. Without wishing to be bound by theory, the increased hydrophobicity of the second bioactive agent blocks some degree of access of the extracellular environment of a subject's sinuses and/or nasal passages to the first bioactive agent, thereby preventing a burst release of the first bioactive agent.

In one aspect, the substrate can be a poly-D/L-lactic acid (PLLA) device. In an alternative aspect, the substrate can be a poly-(D,L-lactide-co-glycolide) (PLGA) device. In still another aspect, the substrate can be made from any of the biocompatible and biodegradable polymers disclosed herein.

The substrate can be any article where that can receive the first and second layers described herein. In one aspect, the substrate comprises an implantable device such as, for example, a stent, a catheter, a nasal/sinus implant, or an intra-sinus tissue implant.

### First Layer

The first layer of the substrate disclosed herein incorporates a first bioactive agent and, in a further aspect, the first bioactive agent can be or include a first antibiotic. In one aspect, the first antibiotic can be a cephalosporin such as, for example, cefazolin, cefuroxime, ceftazidime, cephalexin, cephaloridine, cefamandole, cefsulodin, cefonicid, cefoperazine, cefoprozil, ceftriaxone, or a combination thereof. In another aspect, the antibiotic can be a polymyxin such as, for example, polymyxin B, colistin, or a combination thereof. In still another aspect, the antibiotic can be an aminoglycoside such as, for example, gentamicin, amikacin, tobramycin, debekacin, kanamycin, neomycin, netilmicin, paromomycin, sisomycin, spectinomycin, streptomycin, or a combination thereof. In still another aspect, the antibiotic can be a fluorquinolone such as, for example, levofloxacin, norfloxacin, ofloxacin, ciprofloxacin, perfloxacin, lomefloxacin, fleroxacin, sparfloxacin, grepafloxacin, trovafloxacin, clinafloxacin, gemifloxacin, enoxacin, sitafloxacin, nadifloxacin, tosulfloxacin, cinnoxacin, rosoxacin, miloxacin, moxifloxacin, gatifloxacin, nalidixic acid, nadifloxacin, oxolinic acid, pefloxacin, pirimidic acid, pipernidic acid, rufloxacin, temafloxacin, trovafloxacin, besifloxacin, or any combination thereof. In one aspect, the antibiotic can be a combination of antibiotic agents from multiple classes disclosed above, or another antibiotic. In a further aspect, the first bioactive agent includes ciprofloxacin.

In one aspect, the first bioactive agent is incorporated in a plurality of nanoparticles. Further in this aspect, the nanoparticles incorporate the first antibiotic. In a further aspect, the first antibiotic can be encapsulated by the nanoparticles. In an alternative aspect, the first antibiotic is present on the surfaces of the nanoparticles. In still another aspect, the first antibiotic is both encapsulated by and present on the surfaces of the nanoparticles.

In one aspect, the nanoparticles include a biodegradable and biocompatible polymer. In a further aspect, the polymer can be a polylactide, a polyglycolide, a polylactide-co-glycolide, a polyesteramide, a polyorthoester, a poly-β-hydroxybutyric acid, a polyanhydride, a polydiene, a polyalkylene glycol, a polymethacrylate, a polyvinyl ether, a polyvinyl alcohol, a polyvinyl chloride, a polyvinyl ester, a polycarbonate, a polyester, a cellulose ether, a cellulose ester, a polysaccharide, a polycaprolactone, starch, or any combination thereof.

Polylactic acid is a polyester derived from lactic acid. The polyester is composed of lactic acid units depicted in the structure below, where m indicates the number of lactic acid units. The lactic acid unit has one chiral center, indicated by the asterisk (*) in the structure below:

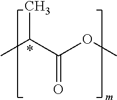

Polylactic acid polymerization can begin from D or L lactic acid or a mixture thereof, or lactide, a cyclic diester. Properties of polylactic acid can be fine-tuned by controlling the ratio of D to L enantiomers used in the polymerization, and polylactic acid polymers can also be synthesized using starting materials that are only D or only L rather than a mixture of the two. Polylactic acid prepared from only D starting materials is referred to as poly-D-lactide (PDLA); conversely, polylactic acid prepared from only L starting materials is poly-L-lactide (PLLA).

As used herein, a D-lactic acid unit or an L-lactic acid unit refers to the monomer units within the polylactic acid polymers described herein, wherein a D-lactic acid unit is derived from the D-lactic acid or D-lactide starting material, and an L-lactic acid unit is derived from the L-lactic acid or L-lactide starting material as shown in Table 1:

TABLE 1
Starting Materials for PDLA and PLLA

| | |
|---|---|
| 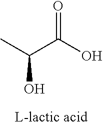 | 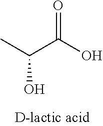 |
| 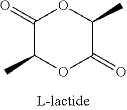 | 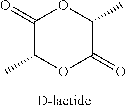 |

In one aspect, the polymer resembles materials used in the manufacture of resorbable synthetic sutures. In another aspect, the polymer is biocompatible and biodegradable within the nasal and sinus passages. In still another aspect, the polymer does not produce any toxic byproducts as it degrades. In still another aspect, the polymer can be modified to modify the duration of drug release by manipulating the polymer's characteristics (e.g., in a polylactide-co-glycolide polymer, modifying the ratio of lactide and glycolide).

In one aspect, the nanoparticles can be prepared by an emulsion/solvent technique. Further in this aspect, a solution of the first bioactive agent in a first solvent can be mixed with a polymer solution in a second solvent. Still further in this aspect, the mixture can be agitated using a method such as, for example, sonication to produce an emulsion. In a further aspect, the emulsion can be stirred into an additional solvent and homogenized to form nanoparticles. In one aspect, the additional solvent can be allowed to evaporate overnight, the nanoparticles can be collected by centrifugation, resuspended in a solvent such as, for example, water, and filtered to remove large particulate matter. In a further aspect, following synthesis of the nanoparticles, properties of the nanoparticles can be determined using techniques including, but not limited to, scanning electron microscopy (SEM), particle size analysis, and determination of zeta potential.

In one aspect, the nanoparticles have a mean diameter of from about 400 to about 700 nm, or of about 400, 425, 450, 475, 500, 525, 550, 575, 600, 625, 650, 675, or about 700 nm, or a combination of any of the foregoing values, or a range encompassing any of the foregoing values. In another aspect, the nanoparticles have a zeta potential of from about 0.1 mV to about 0.5 mV, or of about 0.1, 0.15, 0.2, 0.25, 0.3, 0.35, 0.4, 0.45, or about 0.5 mV, or a combination of any of the foregoing values, or a range encompassing any of the foregoing values.

In another aspect, the nanoparticles incorporating a first antibiotic can be further dispersed in a first polymer. In a further aspect, the nanoparticles are homogeneously dispersed in the first polymer. In a still further aspect, the first polymer is biocompatible. In another aspect, the first polymer can also be biodegradable. In still another aspect, the first polymer can be a homopolymer or a copolymer of an acrylate, a methacrylate, an acrylamide, a methacrylamide, acrylic acid, methacrylic acid, an acrylic acid ester, a methacrylic acid ester, or any combination thereof.

In one aspect, the first layer of the substrate, that includes the nanoparticles, first bioactive agent, and first polymer has a thickness from about 50 to about 200 μm, or of about 50, 60, 70, 80, 90, 100, 110, 120, 130, 140, 150, 160, 170, 180, 190, or of about 200 μm, or a combination of any of the foregoing values, or a range encompassing any of the foregoing values.

In yet another aspect, the first bioactive agent is an antibiotic and is present in the first layer in an amount of from about 1 μg to about 500 mg, or about 1, 25, 50, 100, 150, 200, 250, 300, 350, 400, 450, 500, 550, 600, 650, 700, 750, 800, 850, 900, or 950 μg or 1, 50, 100, 150, 200, 250, 300, 350, 400, 450, or about 500 mg, or a combination of any of the foregoing values, or a range encompassing any of the foregoing values.

### Second Layer

The second layer of the coated substrates described herein includes a second bioactive agent. In a further aspect, the second bioactive agent can be a cystic fibrosis transmembrane conductance regulator (CTFR) potentiator. In one aspect, the CTFR potentiator can be ivacaftor. In any of these aspects, the second bioactive agent can be present in the second layer in an amount of from about 1 μg to about 500 mg, or about 1, 25, 50, 100, 150, 200, 250, 300, 350, 400, 450, 500, 550, 600, 650, 700, 750, 800, 850, 900, or 950 μg or 1, 50, 100, 150, 200, 250, 300, 350, 400, 450, or about 500 mg, or a combination of any of the foregoing values, or a range encompassing any of the foregoing values.

In another aspect, the second bioactive agent can be or include a second antibiotic. Further in this aspect, the second antibiotic can be present in the amount of from about 5 μg to about 500 mg, or about 5, 25, 50, 100, 150, 200, 250, 300, 350, 400, 450, 500, 550, 600, 650, 700, 750, 800, 850, 900, or 950 μg or 1, 50, 100, 150, 200, 250, 300, 350, 400, 450, or about 500 mg, or a combination of any of the foregoing values, or a range encompassing any of the foregoing values.

In one aspect, the second antibiotic can be a cephalosporin such as, for example, cefazolin, cefuroxime, ceftazidime, cephalexin, cephaloridine, cefamandole, cefsulodin, cefonicid, cefoperazine, cefoprozil, ceftriaxone, or a combination thereof. In another aspect, the antibiotic can be a polymyxin such as, for example, polymyxin B, colistin, or a combination thereof. In still another aspect, the antibiotic can be an aminoglycoside such as, for example, gentamicin, amikacin, tobramycin, debekacin, kanamycin, neomycin, netilmicin, paromomycin, sisomycin, spectinomycin, streptomycin, or a combination thereof. In still another aspect, the antibiotic can be a fluorquinolone such as, for example, levofloxacin, norfloxacin, ofloxacin, ciprofloxacin, perfloxacin, lomefloxacin, fleroxacin, sparfloxacin, grepafloxacin, trovafloxacin, clinafloxacin, gemifloxacin, enoxacin, sitafloxacin, nadifloxacin, tosulfloxacin, cinnoxacin, rosoxacin, miloxacin, moxifloxacin, gatifloxacin, nalidixic acid, nadifloxacin, oxolinic acid, pefloxacin, pirimidic acid, pipernidic acid, rufloxacin, temafloxacin, trovafloxacin, besifloxacin, or any combination thereof. In one aspect, the antibiotic can be a combination of antibiotic agents from multiple classes disclosed above, or another antibiotic. In a further aspect, the second bioactive agent includes azithromycin.

In still another aspect, the second layer contains an amount of a third bioactive agent in addition to the second bioactive agent. In any of these aspects, the third bioactive agent and the second bioactive agent are different compounds. In one aspect, the third bioactive agent is a third antibiotic agent including a cephalosporin such as, for example, cefazolin, cefuroxime, ceftazidime, cephalexin, cephaloridine, cefamandole, cefsulodin, cefonicid, cefoperazine, cefoprozil, ceftriaxone, or a combination thereof. In another aspect, the antibiotic can be a polymyxin such as, for example, polymyxin B, colistin, or a combination thereof. In still another aspect, the antibiotic can be an aminoglycoside such as, for example, gentamicin, amikacin, tobramycin, debekacin, kanamycin, neomycin, netilmicin, paromomycin, sisomycin, spectinomycin, streptomycin, or a combination thereof. In still another aspect, the antibiotic can be a fluorquinolone such as, for example, levofloxacin, norfloxacin, ofloxacin, ciprofloxacin, perfloxacin, lomefloxacin, fleroxacin, sparfloxacin, grepafloxacin, trovafloxacin, clinafloxacin, gemifloxacin, enoxacin, sitafloxacin, nadifloxacin, tosulfloxacin, cinnoxacin, rosoxacin, miloxacin, moxifloxacin, gatifloxacin, nalidixic acid, nadifloxacin, oxolinic acid, pefloxacin, pirimidic acid, pipernidic acid, rufloxacin, temafloxacin, trovafloxacin, besifloxacin, or any combination thereof. In one aspect, the antibiotic can be a combination of antibiotic agents from multiple classes disclosed above, or another antibiotic.

In one aspect, the first layer includes ciprofloxacin and the second layer includes azithromycin. In another aspect, the first layer includes ciprofloxacin and the second layer includes azithromycin and ciprofloxacin.

In one aspect, the second layer of the substrate includes a plurality of nanoparticles, wherein the nanoparticles incorporate the second bioactive agent. Further in this aspect, the second bioactive agent can be encapsulated by the nanoparticles, can be on the surfaces of the nanoparticles, or both.

In another aspect, the nanoparticles of the second layer include a biodegradable and biocompatible polymer. In a further aspect, the polymer can be a polylactide, a polyglycolide, a polylactide-co-glycolide, a polyesteramide, a polyorthoester, a poly-β-hydroxybutyric acid, a polyanhydride, a polydiene, a polyalkylene glycol, a polymethacrylate, a polyvinyl ether, a polyvinyl alcohol, a polyvinyl chloride, a polyvinyl ester, a polycarbonate, a polyester, a cellulose ether, a cellulose ester, a polysaccharide, a polycaprolactone, starch, or any combination thereof. In any of these aspects, certain properties, modifications, and components of the biodegradable and biocompatible polymer have been discussed previously with respect to the polymeric composition of the nanoparticles of the first layer. In another aspect, the nanoparticles of the second layer are synthesized in a similar manner to the nanoparticles of the first layer, although some parameters such as, for example, solvent may be altered to fit the properties of the bioactive agent incorporated into the nanoparticles.

In one aspect, the nanoparticles of the second layer have a mean diameter of from about 400 to about 700 nm, or of about 400, 425, 450, 475, 500, 525, 550, 575, 600, 625, 650, 675, or about 700 nm, or a combination of any of the foregoing values, or a range encompassing any of the foregoing values. In another aspect, the nanoparticles have a zeta potential of from about 0.1 mV to about 0.5 mV, or of about 0.1, 0.15, 0.2, 0.25, 0.3, 0.35, 0.4, 0.45, or about 0.5 mV, or a combination of any of the foregoing values, or a range encompassing any of the foregoing values.

In another aspect, the nanoparticles of the second layer can be dispersed in a second polymer. In a further aspect, the nanoparticles are homogeneously dispersed. In still another aspect, the second polymer can be a biodegradable and biocompatible polymer. In yet another aspect, the second polymer can be a homopolymer or copolymer of an acrylate, a methacrylate, an acrylamide, a methacrylamide, acrylic acid, methacrylic acid, an acrylic acid ester, a methacrylic acid ester, or a combination thereof.

In one aspect, the second layer has a thickness of from about 50 μm to about 500 μm, or about 50, 100, 150, 200, 250, 300, 350, 400, 450, or about 500 μm, or a combination of any of the foregoing values, or a range encompassing any of the foregoing values.

### Example Formulations and Devices

In one aspect, disclosed herein is a substrate as described above, wherein about 80% of the first bioactive agent and the second bioactive agent are released from the substrate over a period of about 21 days. In a further aspect, the second, outer layer may slowly disintegrate over time, allowing additional access by the surrounding medium to the first, inner layer, thereby enabling a slow and sustained release of the first bioactive agent in the first layer as disclosed herein.

In another aspect, the substrate as described herein can be an implantable device such as, for example, a stent, a catheter, a nasal/sinus implant, or an intra-sinus tissue implant. In a further aspect, bi-layered substrates such as those disclosed herein can be used to treat or prevent infection and inflammation in numerous applications.

In one aspect, nanoparticles containing or associated with a bioactive agent as disclosed herein can be suspended in a solution of a polymer and used to coat a stent or other device. In one aspect, for a bilayered substrate, a first polymeric/nanoparticle solution is deposited, following by a second coating with a second polymeric/nanoparticle solution. In a further aspect, the stents or other devices can be dried under vacuum following deposition of either or both of the polymeric/nanoparticle solutions.

### Methods of Use

Chronic bacterial infections with pathogenic organisms that form biofilms (e.g., Pseudomonas aeruginosa) and associated sinonasal inflammatory responses have been identified as common reasons for persistence of recalcitrant chronic inflammatory diseases. Pharmacological interventions for severe disease have been limited to surgical intervention (often repetitive) and systemic+/−topical antibiotic sinus irrigations in attempts to eliminate chronic infections due to these biofilm-forming organisms. The substrates described herein provide an efficient treatment strategy with high translational potential to improve clinical outcomes in chronic inflammatory diseases.

In one aspect, disclosed herein is a method for killing bacteria in a subject or preventing the growth of bacteria in a subject, wherein the method includes the step of administering the substrate as described herein to the subject. In another aspect, disclosed herein is a method for treating or preventing a bacterial infection in a subject, wherein the method includes the step of administering the substrate as described herein to the subject.

In either of the above aspects, the substrate can be administered to the nasal and/or sinus cavities of the subject, including, but not limited to, areas such as the nasopharynx and/or anterior skull-base.

In one aspect, when administered to the subject, the substrate as disclosed herein reduces biofilm mass in the nasal cavity.

In another aspect, when administered to the subject, the substrates described herein can treat or prevent inflammation in the subject. In another aspect, when administered to the subject, the substrates described herein can reduce or prevent the production of interleukin-8 (IL-8) in the subject. Interleukin-8 (IL-8) is chemoattractant cytokine that is upregulated in chronic inflammatory diseases such as, for example, chronic rhinosinusitis (CRS) with increasing levels correlated to higher disease severity (neutrophil infiltration). IL-8 inhibition is considered one of the primary mechanisms of reduced airway inflammation in patients. As demonstrated in the Examples, the substrates described herein are effective in inhibiting the production of IL-8.

In any of the above aspects, the substrate can be administered to or implanted in a subject who has a chronic inflammatory disease of the upper airway system. In still another aspect, the subject can have chronic rhinosinusitis, hyposmia or anosmia, chronic rhinitis, allergic rhinitis, vasomotor rhinitis, another inflammatory respiratory disease, or multiple inflammatory respiratory diseases at the same time. In one aspect, the inflammatory disease or infection is caused by Pseudomonas aeruginosa or a related bacterium.

### Aspects

Aspect 1: A substrate comprising a first surface, wherein a first layer comprising a first bioactive agent is adjacent to the first surface of the substrate, and a second layer comprising a second bioactive agent is adjacent to the first layer, wherein the second bioactive agent is more hydrophobic than the first bioactive agent.

Aspect 2: The substrate of Aspect 1, wherein the first bioactive agent comprises an antibiotic.

Aspect 3: The substrate of Aspect 1, wherein the first bioactive agent comprises ciprofloxacin, levofloxacin, norfloxacin, ofloxacin, perfloxacin, lomefloxacin, fleroxacin, sparfloxacin, grepafloxacin, trovafloxacin, clinafloxacin, gemifloxacin, enoxacin, sitafloxacin, nadifloxacin, tosulfloxacin, cinnoxacin, rosoxacin, miloxacin, moxifloxacin, gatifloxacin, nalidixic acid, nadifloxacin, oxolinic acid, pefloxacin, pirimidic acid, pipernidic acid, rufloxacin, temafloxacin, trovafloxacin, besifloxacin, or any combination thereof.

Aspect 4: The substrate in any one of Aspects 1-3, wherein the first bioactive agent is ciprofloxacin.

Aspect 5: The substrate in any one of Aspects 1-4, wherein the first bioactive agent comprises a plurality of nanoparticles, wherein the nanoparticles comprise the first antibiotic.

Aspect 6: The substrate of Aspect 5, wherein the nanoparticles comprise a biodegradable and biocompatible polymer.

Aspect 7: The substrate of Aspect 5, wherein the nanoparticles comprise a polylactide, a polyglycolide, a polylactide-co-glycolide, a polyesteramide, a polyorthoester, a poly-3-hydroxybutyric acid, a polyanhydride, a polydiene, a polyalkylene glycol, a polymethacrylate, a polyvinyl ether, a polyvinyl alcohol, a polyvinyl chloride, a polyvinyl ester, a polycarbonate, a polyester, a cellulose ether, a cellulose ester, a polysaccharide, a polycaprolactone, starch, or any combination thereof.

Aspect 8: The substrate in any one of Aspects 5-7, wherein the nanoparticles have a mean diameter of from about 400 nm to about 700 nm.

Aspect 9: The substrate in any one of Aspects 1-8, wherein the first layer comprises a plurality of nanoparticles, wherein the nanoparticles particles are homogeneously dispersed in a first polymer.

Aspect 10: The substrate of Aspect 9, wherein the first polymer comprises a biodegradable and biocompatible polymer.

Aspect 11: The substrate of Aspect 9, wherein the first polymer comprises a homopolymer or a copolymer of an acrylate, a methacrylate, an acrylamide, a methacrylamide, acrylic acid, methacrylic acid, an acrylic acid ester, a methacrylic acid ester, or any combination thereof.

Aspect 12: The substrate in any one of Aspects 1-11, wherein the first layer has a thickness of from about 50 μm to about 200 μm.

Aspect 13: The substrate in any one of Aspects 1-11, wherein the first bioactive agent is an antibiotic present in the first layer in the amount of about 1 μg to 500 mg.

Aspect 14: The substrate in any one of Aspects 1-13, wherein the second bioactive agent comprises a cystic fibrosis transmembrane conductance regulator (CFTR) potentiator.

Aspect 15: The substrate of Aspect 14, wherein the second bioactive agent comprises ivacaftor.

Aspect 16: The substrate of Aspects 14 or 15, wherein the second bioactive agent is present in the second layer in the amount of about 1 μg to 500 mg.

Aspect 17: The substrate in any one of Aspects 1-16, wherein the second bioactive agent comprises a second antibiotic.

Aspect 18: The substrate of Aspect 17, wherein the second antibiotic is present in the second layer in the amount of about 5 μg to 500 mg.

Aspect 19: The substrate of Aspects 17 or 18, wherein the second antibiotic comprises azithromycin.

Aspect 20: The substrate of Aspect 19, wherein the first bioactive agent comprises ciprofloxacin.

Aspect 21: The substrate in any one of Aspects 1-20, wherein the second layer further comprises a third bioactive agent comprising a third antibiotic.

Aspect 22: The substrate of Aspect 21, wherein the third antibiotic comprises ciprofloxacin.

Aspect 23: The substrate in any one of Aspects 1-22, wherein the second layer comprises a plurality of nanoparticles, wherein the nanoparticles comprise the second bioactive agent.

Aspect 24: The substrate of Aspect 23, wherein the nanoparticles comprise a biodegradable and biocompatible polymer.

Aspect 25: The substrate of Aspect 24, wherein the nanoparticles comprise a polylactide, a polyglycolide, a polylactide-co-glycolide, a polyesteramide, a polyorthoester, a poly-β-hydroxybutyric acid, a polyanhydride, a polydiene, a polyalkylene glycol, a polymethacrylate, a polyvinyl ether, a polyvinyl alcohol, a polyvinyl chloride, a polyvinyl ester, a polycarbonate, a polyester, a cellulose ether, a cellulose ester, a polysaccharide, a polycaprolactone, starch, or any combination thereof.

Aspect 26: The substrate in any one of Aspects 23-25, wherein the nanoparticles have a mean diameter of from about 400 nm to about 700 nm.

Aspect 27: The substrate in any one of Aspects 1-26, wherein the second layer comprises a plurality of nanoparticles, wherein the nanoparticles particles are homogeneously dispersed in a second polymer.

Aspect 28: The substrate of Aspect 27, wherein the second polymer comprises a biodegradable and biocompatible polymer.

Aspect 29: The substrate of Aspect 27, wherein the second polymer comprises a homopolymer or a copolymer of an acrylate, a methacrylate, an acrylamide, a methacrylamide, acrylic acid, methacrylic acid, an acrylic acid ester, a methacrylic acid ester, or any combination thereof.

Aspect 30: The substrate in any one of Aspects 1-29, wherein the second layer has a thickness of from about 50 μm to about 500 μm.

Aspect 31: The substrate in any one of Aspects 1-29, wherein the first bioactive agent comprises a plurality of nanoparticles, comprising a biodegradable and biocompatible polymer and a first antibiotic, and the second bioactive agent comprises a plurality of nanoparticles, comprising a biodegradable and biocompatible polymer and a second antibiotic.

Aspect 32: The substrate in any one of Aspects 1-31, wherein about 80% of the first antibiotic and the second bioactive agent are released from the substrate by 21 days.

Aspect 33: The substrate in any one of Aspects 1-32, wherein the substrate comprises an implantable device.

Aspect 34: The substrate of Aspect 33, wherein the substrate comprises a biodegradable and biocompatible polymer.

Aspect 35: The substrate in any one of Aspects 1-34, wherein the substrate comprises a stent, a catheter, a nasal/sinus implant, or an intra-sinus tissue implant.

Aspect 36: A method for killing bacteria in a subject or preventing the growth of bacteria in a subject, comprising administering to the subject the substrate in any one of Aspects 1-35.

Aspect 37: A method for treating or preventing a bacterial infection in a subject comprising administering to the subject the substrate in any one of Aspects 1-35.

Aspect 38: A method for treating or preventing inflammation in a subject comprising administering to the subject the substrate in any one of Aspects 1-35.

Aspect 39: A method for reducing or preventing the production of interleukin-8 in a subject comprising administering to the subject the substrate in any one of Aspects 1-35.

Aspect 40: The method in any one of Aspects 36-39, wherein the substrate is administered to the nasal and sinus cavity of the subject including nasopharynx and anterior skull-base.

Aspect 41: The method in any one of Aspects 36-40, wherein the substrate reduces biofilm mass in the nasal cavity.

Aspect 42: The method in any one of Aspects 36-41, wherein the subject has chronic inflammatory disease in the upper airway system.

Aspect 43: The method in any one of Aspects 36-41, wherein the subject has chronic rhinosinusitis, hyposmia/anosmia, chronic rhinitis, allergic rhinitis, or vasomotor rhinitis.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. Numerous variations and combinations of reaction conditions (e.g., component concentrations, desired solvents, solvent mixtures, temperatures, pressures, and other reaction ranges and conditions) can be used to optimize the product purity and yield obtained from the described process. Only reasonable and routine experimentation will be required to optimize such process conditions.

### Example 1: Materials

Ciprofloxacin HCl (99.5% purity) was purchased from GenHunter Corporation (Nashville, Tenn.). Ivacaftor (VX-770) was obtained from Selleckchem (Houston, Tex.). Azithromycin was purchased from TCI America (Portland, Oreg.). Poly (D,L-lactide-co-glycolide) (PLGA) was purchased from PolySciTech (West Lafayette, Ind.). All other chemicals and reagents used in this study were purchased from Sigma-Aldrich (St. Louis, Mo.).

### Example 2: Preparation of PLGA Nanoparticles with Ciprofloxacin and Ivacaftor

### Ciprofloxacin-Loaded PLGA Nanoparticles

An emulsion/solvent technique was used to create ciprofloxacin-loaded PLGA nanoparticles. First, 15 mg of ciprofloxacin was dissolved in 750 μL deionized water and subsequently mixed with 2.5% (w/v) of PLGA solution in dichloromethane (DCM). This solution was then placed in an iced water bath and ultrasonically agitated to prepare a homogenous water in oil emulsion (W/O). For each batch, 1 mL of the resulting W/O emulsion was immediately poured into 25 mL of a 1% (w/v) polyvinyl alcohol solution (PVA, 98.0˜98.8% hydrolyzed, M_{w}=31,000-50,000) and stirred vigorously for 2 min using a homogenizer. The suspended nanoparticles were then placed onto a stir plate to evaporate the organic solvent in a chemical fume hood overnight. The nanoparticles were then collected by centrifugation at 4000 rpm for 20 min at 4° C., and resuspended in 10 mL of distilled water, following which large particulates and aggregates were removed through the use of a 40 μm cell strainer.

### Ivacaftor-Loaded PLGA Nanoparticles

Ivacaftor-containing nanoparticles were fabricated using a solvent evaporation method in a similar fashion to ciprofloxacin nanoparticles. An ivacaftor stock solution (25 mg/mL) was prepared in dimethyl sulfoxide (DMSO) and aliquoted into separate 300 μL ivacaftor solutions. To prepare a mixture of ivacaftor and PLGA in DCM, the 300 μL ivacaftor solution was dissolved in 10 mL of dichloromethane containing 250 mg of PLGA polymer. Subsequently, 1 mL of the mixture was slowly added into 25 mL of 1% (w/v) PVA solution, and then homogenized at 32,000 rpm for 2 min to create ivacaftor-containing nanoparticles. After creating ivacaftor-loaded PLGA nanoparticles, the collection was made based on similar protocols of ciprofloxacin-loaded PLGA nanoparticles, described above.

### Example 3: Characterization of the Prepared PLGA Nanoparticles with Ciprofloxacin or Ivacaftor

To examine the morphology of the fabricated PLGA nanoparticles, a field emission scanning electron microscope (SEM) was used (FE-SEM, Quanta FEG-650, USA). Prior to scanning, the PLGA nanoparticles were coated with an Au—Pd sputter to enhance surface conductivity while reducing charging artifacts. An accelerating voltage of 20 kV was used in most cases, and the SEM images were processed at the UAB SEM Laboratory. Zeta potentials (the electrostatic potential at the electrical double layer surrounding a nanoparticle in solution) were determined by using a Malvern Zetasizer Nano ZS apparatus (Malvern Instruments Ltd, Worcestershire, UK).

### Surface Morphology and Size of Drug-Loaded PLGA Nanoparticles

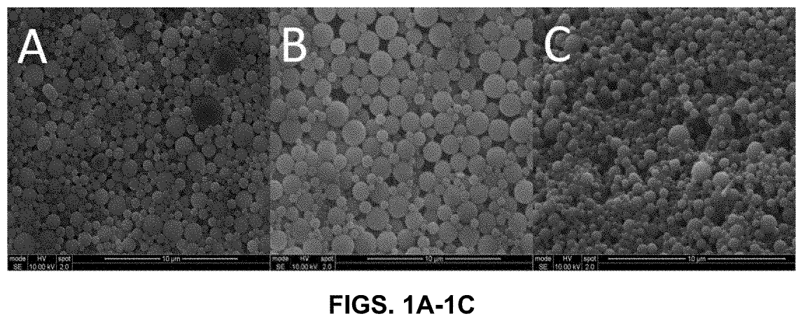

To visualize the surface morphology and size distribution, nanoparticles loaded with ciprofloxacin or ivacaftor were imaged with SEM. Drug-loaded nanoparticles were spherical in shape (FIGS. 1A-1C). In terms of particle size, both nanoparticle configurations had similar size distributions (mean diameter of ciprofloxacin nanoparticle=556.6+/−64.05 nm, mean diameter of ivacaftor nanoparticle=553.8+/−72.93 nm, p>0.05). The measurement of zeta potential was performed to confirm the surface charge property of the nanoparticles formulations, which identifies the presence of ciprofloxacin or ivacaftor within the PLGA nanoparticles. The zeta potential of the empty PLGA nanoparticles was −1.28+/−1.3 mV, a negative charge, indicating the lack of any drug present on the surface of the nanoparticles. In contrast, drug loaded nanoparticles produced a higher, positive charge, with ciprofloxacin-loaded particles exhibiting a zeta potential of 0.17+/−1.1 mV (p<0.01), and ivacaftor-loaded particles exhibiting a zeta potential of 0.27+/−1.4 mV (p=0.005). These results confirmed the presence of ciprofloxacin and ivacaftor on the PLGA nanoparticles.

### Example 4: Fabrication of a Ciprofloxacin- and Ivacaftor-Coated Sinus Stent for In Vitro Analysis

Model biodegradable poly-D/L-lactic acid (PLLA) stents (Biogeneral, Inc. San Diego Calif.) were utilized to create the CISS. Two separate layers of coating were generated: 1) inner layer—ciprofloxacin only and 2) outer layer—ciprofloxacin+ivacaftor. The inner layer (ciprofloxacin nanoparticles) was first coated in the model PLLA stents with a solution of Eudragit RS 100 polymer in acetone (25% w/v). Eudragit RS 100 (a copolymer of ethyl acrylate, methyl methacrylate and a low content of methacrylic acid ester with quaternary ammonium groups) was used for time-controlled drug release by sustained release formulations. Ciprofloxacin nanoparticles were coated in the inner layer to prevent burst release of hydrophilic ciprofloxacin. In the outer layer, the stents were coated with both ciprofloxacin and ivacaftor nanoparticles suspended in the solution of Eudragit RS 100. Sixty μg of ciprofloxacin and 300 μg of ivacaftor were coated on the stent. Finally, the stents were dried under vacuum for 2 days at room temperature.

### Structural Morphology of the CISS

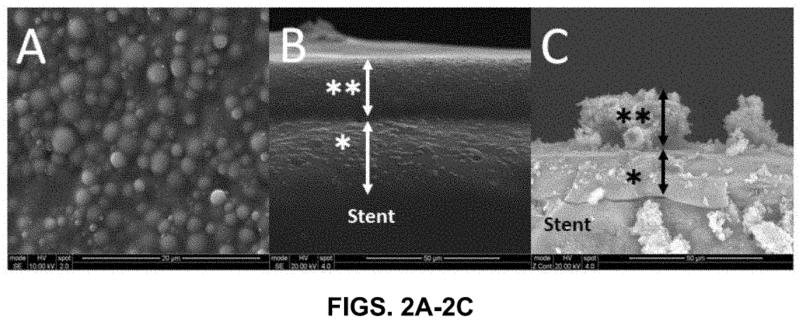

The surface morphology of the CISS was characterized using SEM (FIGS. 2A-2C). Both ciprofloxacin and ivacaftor loaded nanoparticles were embedded within an acrylate and ammonium methacrylate copolymer polymeric matrix coating on the surface of the PLLA stents. A top-down view of the CISS (FIG. 2A) shows the presence of nanoparticles within the coating matrix produced a “bumpy,” mountainous appearance with size distribution approaching a similar size to nanoparticle images (FIG. 1A-1C). Cross sectional images (FIG. 2B) of the CISS demonstrated that two layers of the nanoparticles are present throughout the full thickness of the coating. Additionally, both inner and outer layers can be easily visualized with clear distinction. To understand the degradation of the CISS over time, cross-sectional SEM imaging was performed on stents used in the drug release profile experiment at 21 days (FIG. 2C). There was a bulk loss of the outer layer over the course of the experiment, with thinning of both layers present on the stent surface and absence of the “bumpy” appearance. This confirms degradation of the coated layers with the nanoparticles released over time.

### Example 5: In Vitro Release Profile of the CISS

To assess their in vitro release profiles, model CISS stents containing ciprofloxacin (60 μg) and ivacaftor (300 μg) were placed in 3 mL of sterilized phosphate buffered saline (PBS) and collected periodically for up to 21 days. For the assay of released ciprofloxacin concentration, a ciprofloxacin enzyme-linked immunosorbent assay (ELISA) kit (REAGEN™, Moorestown N.J.) was used according to the manufacture's protocol. The ivacaftor concentration was evaluated by measuring the absorbance at 230 nm using a microplate reader (Synergy HK, BIO-TEK Instruments, Winooski, Vt.).

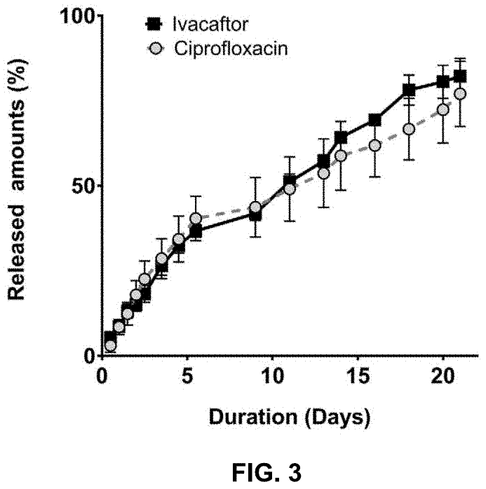

To determine the specific release profile for the drug eluting stent, an in-vitro release profile assay was performed (FIG. 3). A similar steady-state release was achieved with both drugs without an initial burst release. Approximately 50% of the coated ciprofloxacin and ivacaftor were released by 10 days (ciprofloxacin 49.1+/−9.4%, ivacaftor 51.2+/−2.3% at day 10) and 80% by 21 days (ciprofloxacin 77.1+/−9.6%, ivacaftor 82.2+/−5.3% at day 21). Although ivacaftor was only present on the outer layer of the CISS stent, a sustained release profile over the course of the 21 days was identified, The hydrophobic nature of the ivacaftor is likely responsible for the consistent release.

### Example 6: Evaluation of Anti-Biofilm Activity of CISS

### Quantitative Analysis by Crystal Violet Staining

To assess the efficacy of the fabricated CISS against P. aeruginosa (PAO-1 strain) biofilms, a crystal violet assay was used. Briefly, stents loaded with the drugs were placed in a 24-well tissue culture plate and inoculated with 100 μL of 100-fold diluted overnight culture grown at 30° C. Stents without loaded drugs served as negative controls. After 3 days, the attached biofilm was assessed as previously described. Three ml of 0.1% (w/v) crystal violet was used to stain the biofilms. Next, 900 μL of 30% acetic acid was used to dissolve the PAO-1 biofilms and release the conjugated crystal violet dye. Absorbance was then measured at 590 nm to quantify the amount of crystal violet present.

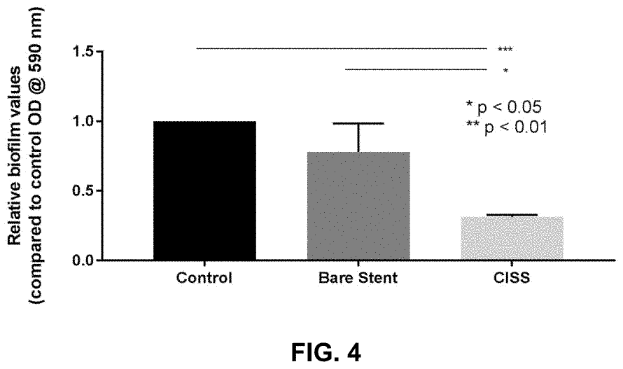

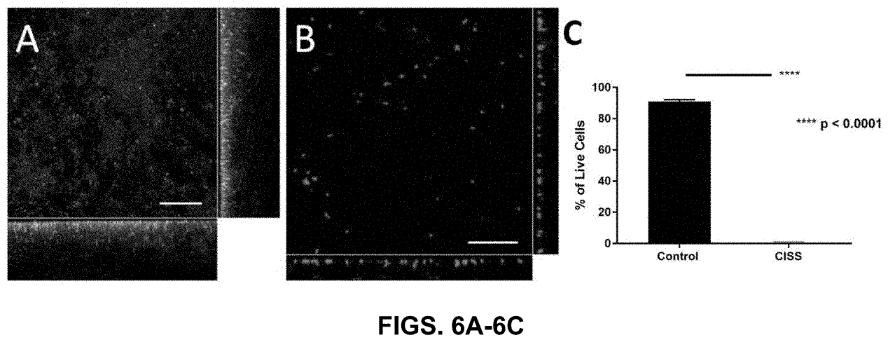

To determine the anti-biofilm activity of CISS stents, a standard crystal biofilm assay was performed (FIGS. 6A-6C). In this assay, PAO-1 biofilms were grown for 24 hours and then subjected to 1 of the following 3 conditions for 72 hours: 1) CISS, 2) PLLA stent without drugs (bare stent), 3) control. The CISS significantly reduced biofilm mass compared to bare stents and controls (relative biofilm value compared to control at OD₅₉₀, CISS=0.31+/−0.01, bare stent=0.78+/−0.12, control=1.0+/−0.00, p=0.001, n=3) (FIG. 4).

### Quantitative Analysis by Confocal Laser Scanning Microscopy (CLSM)

To create pre-formed PAO-1 biofilms, PAO-1 was cultured for 24 hours on 14 mm glass coverslips within a 35 mm dish (MatTek, Ashland, Mass.). In the pre-formed PAO-1 biofilms, stents containing loaded drugs (ciprofloxacin and ivacaftor) were then placed in a 24-well tissue culture plate and cultured for an additional three days. Stents without loaded drugs were also introduced to serve as a negative control. To visualize both viable and dead bacterial populations, biofilms were stained with SYTO9 and propidium iodide (PI) staining (BacLight™ Live/Dead Bacterial Viability Kit; Molecular Probes, Eugene, Oreg.). The biofilms were imaged with CLSM (A1R, Nikon, Tokyo, Japan) and biofilm thickness quantified using Image J by referencing at least 4 different images per condition. The proportions of live and dead bacteria were also quantified using BioFilmAnalyzer v.1.0 by counting fluorescence specific pixels in digital fluorescent images. Five different images were selected for analysis per condition.

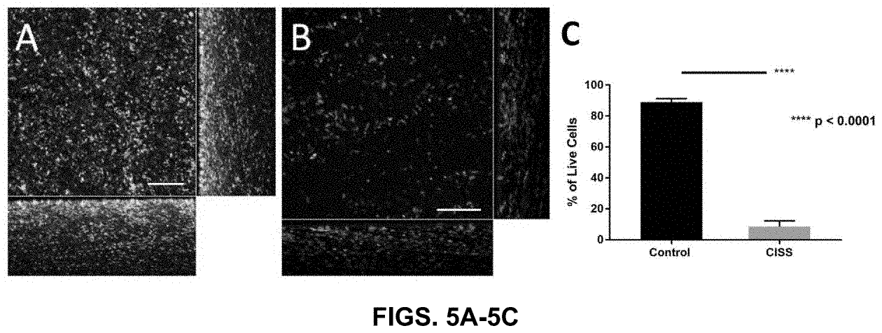

The bactericidal efficacy of CISS stents against P. aeruginosa biofilms was further assessed using live-dead staining. To evaluate whether CISS stents can prevent the formation of biofilms, bacteria were grown in the presence of CISS stents and biofilm height was measured on day 1. Biofilm thickness was significantly lower in the presence of CISS (11.66+/−2.8 μm) compared to without CISS (22.94+/−3.30 μm), indicating that CISS stents can inhibit the formation of PAO-1 biofilms (p<0.01) (FIGS. 5A-5C). Biofilm formation was significantly decreased in the presence of CISS compared to controls without CISS (% of live cells; control without CISS=88.9+/−2%; CISS=8.5+/−3.2%; n=4 per condition, p<0.0001) (FIG. 5C). To assess the capability of the stent to eradicate biofilms (efficacy), the CISS was placed on pre-formed 1-day old PAO-1 biofilms and grown for additional 3 days (FIGS. 6A-6C). There was a significant reduction in the PAO-1 biofilm height (4.16+/−1.87 μm) when compared to those from controls without exposure to the CISS (14.94+/−2.78 μm) (p<0.001). Biofilm presence was significantly decreased in the presence of CISS on day 4 compared to controls without CISS (% of live cells; control without CISS=91.1+/−1%; CISS=0.3+/−0.6%; n=4 per condition, p<0.0001) (FIG. 6C). Biofilms within the control group also had a mixture of both live and dead cells likely due to nutrient deprivation from over confluence of bacteria.

### Example 7: Fabrication of a Ciprofloxacin-Azithromycin Sinus Stent (CASS) for In Vitro Analysis

### Ciprofloxacin-Nanoparticle Suspension for the Inner Layer Coating of the CASS

To create a coating solution containing ciprofloxacin for the inner layer, a nano-precipitation method was developed. First, the aqueous ciprofloxacin solution was prepared by dissolving 40 mg of ciprofloxacin into 1.5 mL of deionized water. Separately, Eudragit RS 100 (a copolymer of ethyl acrylate, methyl methacrylate, and a low content of methacrylic acid ester with quaternary ammonium groups) were dissolved into acetone to prepare a 35% Eudragit RS 100 solution. Then 1.5 ml of the aqueous ciprofloxacin solution and 1.5 ml of 3 5% Eudragit RS 100 solution were mixed and sonicated for 30 minutes to obtain a ciprofloxacin-nanoparticle suspension. Eudragit RS 100 has commonly been used in sustained-release pharmaceutical formulations to encourage a longer lasting effect.

### Azithromycin Polymeric Solution for the Outer Layer Coating of the CASS

The outer layer coating solution was composed of an acrylate/ammonium methacrylate copolymer (Eudragit RL 100, Evonik) and azithromycin, which is a hydrophobic and ethanol-soluble molecule. To create an outer coating solution, 40 mg of azithromycin was dissolved into 1.5 mL of absolute alcohol and mixed with 1.5 mL of the 35% acrylates/ammonium methacrylate copolymer solution.

Coating Ciprofloxacin-Nanoparticle Suspension and Azithromycin Solution onto Biodegradable Poly-D/L-Lactic Acid (PLLA) Stents

To create the CASS, model biodegradable poly-D/L-lactic acid (PLLA) stents (Biogeneral, Inc., San Diego Calif.) were utilized in this study. Dual coating layers were fashioned onto the PLLA stents. First, the inner layer was coated with the ciprofloxacin-nanoparticle suspension. The stents were completely dried and placed in a vacuum for further coating processing. Next, the azithromycin-containing solution was used to create the outer layer. Coated CASSs were subject to an additional drying process in a vacuum for 2 days at room temperature. Sixty μg of ciprofloxacin was coated in the inner layer, while 3 mg of azithromycin was incorporated into the final CASS.

### Structural Morphology of the CASS

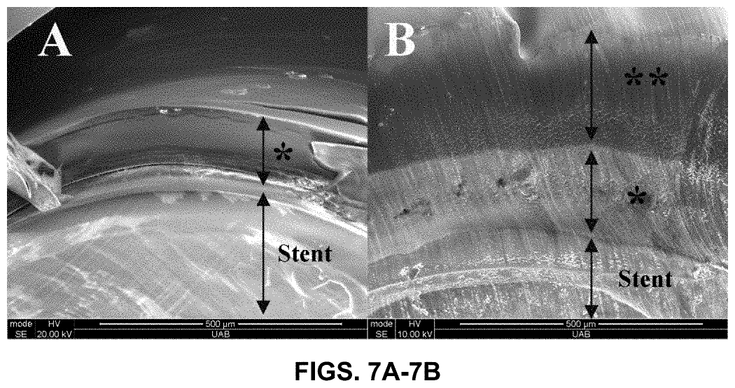

To examine the dual coated structure of the proposed CASS, a cross-sectional view of the CASS was imaged using scanning electron microscopy (SEM) (FIGS. 7A-7B). The ciprofloxacin-nanoparticle suspension was initially embedded within an acrylate and ammonium methacrylate copolymer polymeric matrix to create the inner layer on the PLLA stent surface (FIG. 7A). When characterized by a zeta potential, the ciprofloxacin-nanoparticle suspensions were measured as +45.27+/−0.87 mV. Since ciprofloxacin is a negatively charged compound, the overall positive zeta-potential values demonstrated that the ciprofloxacin was encapsulated within the positively charged acrylates/ammonium methacrylate copolymer. Using an image analysis of SEM, the average thickness of the inner layer was observed as 120.9+/−4.9 μm. The average thickness of the outer layer was 256.2+/−14.60 μm, which was about twice of that of the inner layer (FIG. 7B). Cross sectional images (FIG. 7B) of the CASS demonstrates that the outer layers can be distinguished from the inner layer.

### Example 8: In Vitro Release Profile of the CASS

For assessing the ciprofloxacin and azithromycin release kinetics in the CASS, two different groups of stents were prepared: 1) single ciprofloxacin coated stents and 2) dual coated stents containing ciprofloxacin in the inner layer and azithromycin in the outer layer. All of the samples (n=3 in each group) were incubated in 4 mL of sterilized phosphate buffered saline (PBS) at 37° C. for up to 28 days, and were subject to a periodic collection. To measure the released ciprofloxacin concentration, a ciprofloxacin enzyme-linked immunosorbent assay (ELISA) kit (REAGENT™, Moorestown N.J.) was used according to the manufacture's protocol. The azithromycin concentration was assessed by a spectrophotometric method, as described previously, with a slight modification. This protocol was based on the reduction of potassium permanganate in alkaline solution in the presence of azithromycin. 200 μL of potassium permanganate (0.012 M) solution and 200 μL of potassium carbonate (0.1 M) solution were mixed, and subsequently 200 μL of a collected sample was added. Deionized water was added to make 2 ml of final solution and mixed thoroughly. The absorbance of samples was measured at 547 nm using a microplate reader (Synergy HK, BIO-TEK Instruments, Winooski, Vt.).

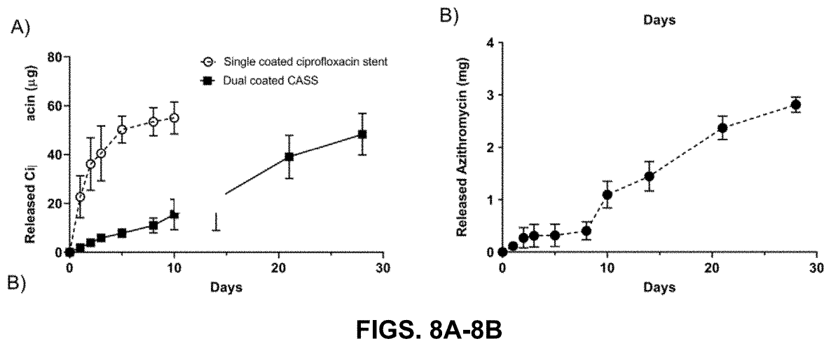

To demonstrate the ability of the dual coated stent to provide sustained release of ciprofloxacin, the release kinetics of ciprofloxacin (60 μg) and azithromycin (3 mg) from the CASS group was compared to that of a single coated ciprofloxacin stent (FIG. 8A). Single coated stents with ciprofloxacin only exhibited a burst release pattern over 10 days. 60.16+/−14.65% of coated ciprofloxacin was released by 2 days, and 83.81+/−7.51% by 5 days. At 10 days, nearly 100% of the coated ciprofloxacin was eluted. In contrast, the dual coated CASS group demonstrated a sustained release of ciprofloxacin over a 28-day period. Briefly, 25.84+/−8.47% of coated ciprofloxacin in the inner layer was released by 10 days, and 65.11+/−12.05% by 21 days. At 28 days, most of the drug was released (80.55+/−11.61%). Azithromycin also had sustained release throughout the study as follows: week 1=0.064±0.061 mg/day, week 2=0.173±0.026 mg/day, week 3=0.132±0.012 mg/day and week 4=0.063±0.015 mg/day (FIG. 8B).

### Example 9: Evaluation of Anti-Biofilm Activity of CASS

### Quantitative Analysis by Crystal Violet Staining

Based on previous work, a crystal violet assay was used to assess the efficacy of the CASS against P. aeruginosa (PAO-1 strain) biofilms. Stents loaded with drugs were placed in a 48-well tissue culture plate. The stents were placed into Luria-Bertani (LB) media and then inoculated with 1×10⁶ PAO-1. Stents without loaded drugs (bare stents) served as negative controls. After 3 days, the attached biofilm was assessed as previously described. 900 μL of 0.1% (w/v) crystal violet was used to stain the biofilms. Next, 900 μL of 30% acetic acid was used to dissolve the PAO-1 biofilms and release the conjugated crystal violet dye. Absorbance was measured at 590 nm to quantify the amount of crystal violet present.

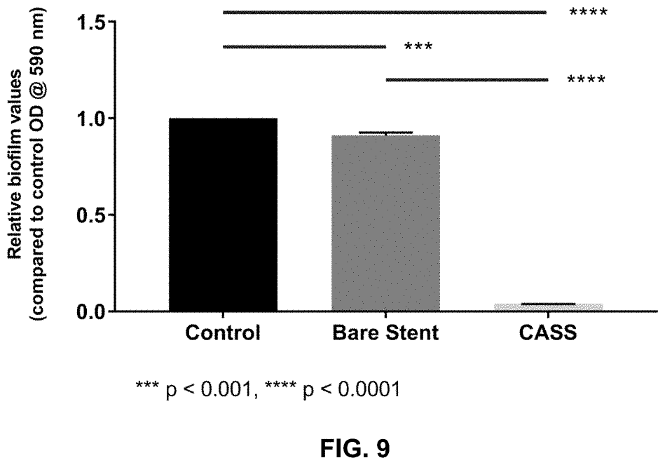

A standard crystal biofilm assay was used to measure the anti-biofilm activities of CASS. To determine the inhibition of P. aeruginosa PAO-1 biofilms by the CASS, 4 conditions were studied: 1) CASS, 2) bare stent (which is a PLLA stent without coating), and 3) control (FIG. 9). After inoculating 1×10⁶ PAO-1 in each condition, P. aeruginosa PAO-1 biofilms were developed for 72 hours and then subjected to crystal violet staining. The CASS significantly decreased P. aeruginosa PAO-1 biofilm mass compared to other conditions. Relative biofilm values calculated by relative optical density units (RODUs) were CASS=0.037+/−0.006, bare stent=0.911+/−0.015, control=1.000+/−0.000 (p<0.001, n=3).

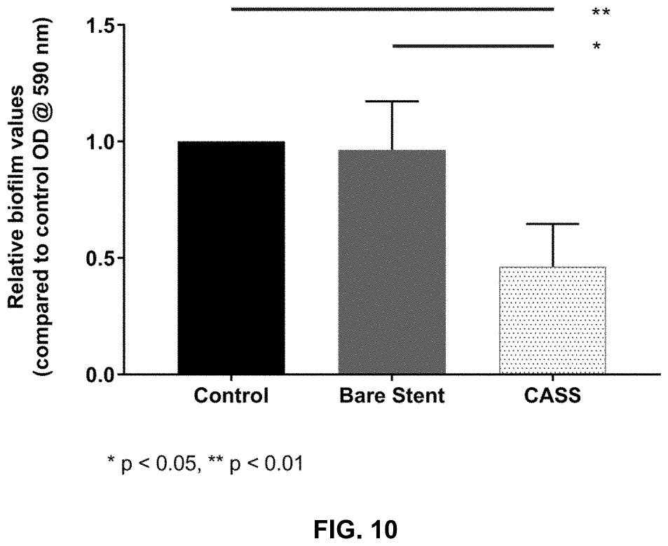

To evaluate the eradication of PAO-1 biofilm by CASS, PAO-1 biofilms were made by inoculating the 1×10⁶ PAO-1 into LB media and cultivating them for 24 hours. 3 groups were subject to the following experiments. Samples were placed into the preformed PAO-1 biofilms and cultured for an additional 3 days. The 3 groups were 1) CASS, 2) bare stent (which is a PLLA stent without coating), and 3) control (FIG. 10). Relative biofilm values compared to control at OD₅₉₀ were CASS=0.463+/−0.183, bare stent=0.964+/−0.209, and control=1.000+/−0.000 (p<0.01, n=3, respectively). As expected, the CASS group exhibited a significant reduction in biofilm mass compared to controls.

### Quantitative Analysis by Confocal Laser Scanning Microscopy (CLSM)

To create pre-formed PAO-1 biofilms, PAO-1 was cultured for 24 hours on 14 mm glass coverslips within a 35 mm dish (MatTek, Ashland, Mass.). In the pre-formed PAO-1 biofilms, stents containing loaded drugs (ciprofloxacin and azithromycin) were placed in a 24-well tissue culture plate and cultured for an additional 3 days. Stents without loaded drugs were also introduced to serve as a negative control. To visualize both viable and dead bacterial populations, biofilms were stained with SYTO9 and propidium iodide (PI) (BacLight™ Live/Dead Bacterial Viability Kit; Molecular Probes, Eugene, Oreg.). The biofilms were imaged with CLSM (A1R, Nikon, Tokyo, Japan), and quantified with a built-in software. The proportions of live and dead bacteria were also quantified using BioFilmAnalyzer v.1.0 by counting fluorescence specific pixels in digital fluorescent images. Four different images per condition were selected for analysis.

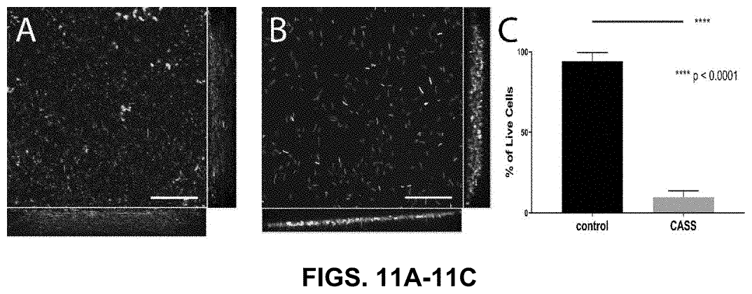

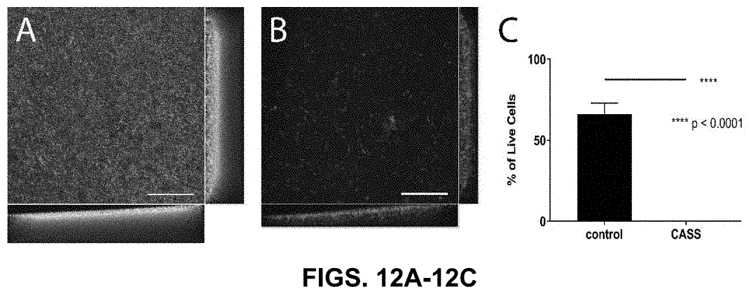

The CASS were placed in the P. aeruginosa inoculated media for 1 day to demonstrate their efficacy against PAO-1 biofilms (FIGS. 11A-11C). When cultured with the CASS, the thickness of biofilms was significantly reduced; 21.60+/−3.94 μm, as compared to 29.63+/−1.47 μm in the controls (p=0.0062), indicating that the CASS stent successfully inhibited their formation. Based on image analysis, the percentage of living PAO-1 cells in the CASS was significantly decreased (% of live cells; control without CASS=94.19+/−5.37% and CASS=9.86+/−3.95%; n=3 per condition, p<0.0001) (FIG. 12C).

To assess the ability of the CASS to eradicate preformed biofilms, CASS were placed on 1-day old PAO-1 biofilms and cultured for additional 3 days (FIGS. 12A-12C). There was a marked reduction in living PAO-1 cells in the biofilm mass in the presence of CASS. After 4 days, the percentage of living cells with CASS was 00.00+/−00.00% whereas control without CASS represented 66.19+/−6.73%, of living PAO-1 cells (p<0.0001, n=3). In addition, P. aeruginosa PAO-1 biofilm mass was significantly reduced by the CASS stents. The PAO-1 biofilm height with CASS (14.7+/−0.76 μm) was markedly lower than those from controls (44.68+/−5.24 μm) (p=0.001). However, it should be noted that the control had a significant number of dead cells because of nutrient deprivation during the 4-day period of cultivation.

### Example 10: Statistical Analysis

All experiments were performed in triplicate. Statistical analysis was performed with GraphPad Prism 6.0 (La Jolla, Ca). For assessing the anti-biofilm activity of stents, a one-way ANOVA was performed with a post-hoc Dunnett's multiple comparison test. Significance was set at p<0.05. Normalized values for relative biofilm quantification were expressed as ±standard error of the mean. For analyzing the difference between control and stents in the CLSM, t-tests were performed.

### Example 11: Evaluation of Azithromycin and Ciprofloxacin for the Inhibition of Interleukin-8 Secretion

### Materials and Methods

Materials and Tested Concentrations

Azithromycin was obtained from TCI America (Portland, Oreg., USA), and ciprofloxacin HCl (99.5% purity) was purchased from GenHunter Corporation (Nashville, Tenn., USA). All other chemicals and reagents used in this study were purchased from Sigma-Aldrich (St. Louis, Mo., USA). The average dose of ciprofloxacin and azithromycin released daily were 2 μg and 120 μg, respectively. Therefore, the dosages of each drug were adjusted with incubation/exposure duration. For example, we used the 0.5 μg of ciprofloxacin and 30 μg of azithromycin as the duration of exposure was only 6 hours.

HSNEC Cultured at an Air-Liquid Interface (ALI)

Primary HSNECs were collected with prior approval of the Institutional Review Board at University of Alabama at Birmingham (UAB) (IRB-120305017). Written informed consent from each participant was collected and subjects were screened and negative for a mutation in the cystic fibrosis transmembrane conductance regulator gene. Primary HSNECs were derived from turbinate mucosa from 3 healthy donors undergoing surgery for non-inflammatory conditions and cultured on collagen-coated Costar 6.5-mm-diameter permeable filter supports (Corning, Lowell, Mass.) submerged in established culture media. Differentiation and ciliogenesis occurred in all cultures within 10 to 14 days.¹⁷⁻²⁶ Matured HSNECs with resistance over 300 Ω/cm² and 80% field ciliogenesis by inverted microscopy were subjected to experiments.

Interleukin-8 Cytokine (IL-8) Concentrations

IL-8 concentrations in the presence of azithromycin and/or ciprofloxacin were obtained using an enzyme linked immunosorbent assay (ELISA) kit for IL-8 (R&D systems, Minneapolis, Minn.). HSNECs were initially exposed to P. aeruginosa LPS for 2 hours, and then incubated up to 24 hours following treatment with azithromycin, ciprofloxacin, or a combination of these drugs. A dose escalation study of azithromycin (from 60 μg/ml to 180 μg/ml) (week 1=0.064±0.061 mg/day, week 2=0.173±0.026 mg/day, week 3=0.132±0.012 mg/day and week 4=0.063±0.015 mg/day) was performed incubating HSNECs with LPS at the lowest dose 6 μg/ml (10 fold lower than 60 μg/ml). Based on these results, LPS treated HSNECs were incubated with ciprofloxacin and the lowest dose of azithromycin that inhibited IL-8 to evaluate synergy and additive effects. A 2.4 μg/ml dose of ciprofloxacin was used based on our in-vitro releasing studies (2.48±0.46 μg/day on average).¹⁰ In each set of experiments, 100 μL of sterilized phosphate buffered saline (PBS) without P. aeruginosa LPS served as vehicle control. After the incubation period, the basal media (600 μL) were collected and measured for IL-8 concentration in each sample according to the manufacture's protocol (R&D systems, Minneapolis, Minn.). The secreted IL-8 concentration in each sample was evaluated by measuring the absorbance at 450 nm using a microplate reader (Synergy HK, BIO-TEK Instruments, Winooski, Vt.). All samples were performed in triplicate.

Measurement of Transepithelial Electrical Resistance (TEER)

Transepithelial electrical resistance (TEER) of HSNECs was measured using a EVOM2 epithelial volt-ohmmeter (World Precision Instruments, Sarasota, Fla., USA).²⁷ The TEER is a summated value from both transcellular and paracellular resistances. The electrical impedance originating from the transcellular pathway represents the stability of the apical and basolateral plasma membranes, whereas the paracellular resistance is created when adequate cell-substrate or cell-cell contacts are formed in the HSNECs when cultured at an air liquid interface. 100 μL of sterilized PBS or drug-containing PBS was dispensed into the apical chamber of the filter supports to produce an electrical circuit. After confirming the maturity of the cultured HSNECs by resistance and morphological observation under an inverted microscope, the planned experiments were conducted. The concentrations of azithromycin (30 μg/ml) and ciprofloxacin (0.5 μg/ml) and the period of incubation were selected to simulate clinically relevant conditions. For example, 30 μg/ml over the course of the six-hour incubation results in 120 μg/ml of azithromycin per day, a quantity similar to the 108 μg noted in our previous study.¹⁰ Azithromycin (30 μg/ml) or a mixture of azithromycin (30 μg/ml) and ciprofloxacin (0.5 μg/ml) were placed onto the apical surface of the cells. TEER was normalized against the PBS control and expressed as Ohms per square centimeter (Ω/cm²). These normalized values enable us to compare the difference of the epithelial membrane's integrity between control and experimental groups. No disruption of the epithelial membrane in the presence of drugs can be observed as equal or higher than normalized TEER value 1.

In Vitro Diffusion-Barrier Function

To examine changes to the diffusion-barrier of HSNECs, paracellular permeability of model 10-kDa fluorescein isothiocyanate (FITC) labeled dextran was evaluated. The diffusion-barrier of HSNECs is determined by a tight epithelial barrier and tight junction formation and is impacted by a number of chemicals. By adding ciprofloxacin and azithromycin at given concentrations, the effects on altered permeability of 10-kDa FITC dextran can be measured. In brief, 0.5 mg/mL 10 kDa FITC-dextran in 100 μL PBS was administered to the apical side of HSNECs for 6 hours in the presence or absence of ciprofloxacin (0.5 μg/ml) and azithromycin (6 μg/ml). The permeated concentrations of FITC dextran were quantified by a fluorescence microplate reader.

Ciliary Beat Frequency Analysis

To acquire ciliary beat frequency (CBF), the HSNECs were placed under a 20× objective on an inverted scope (Fisher Scientific, Pittsburgh, Pa.) at ambient temperature, and treated with azithromycin (30 μg/ml), ciprofloxacin (0.5 μg/ml), and azithromycin/ciprofloxacin (30 μg/ml and 0.5 μg/ml, respectively). After applying 100 μL of sterilized PBS as control or PBS containing drug to the apical surface of the cells, the motion images were acquired for 30 minutes. Under each condition, image acquisition was performed using a Basler area scan high-speed monochromatic digital video camera (Basler AG, Ahrensburg, Germany) at a sampling rate of 100 frames per second. The Sisson-Ammons Video Analysis (SAVA) system was used to acquire CBF. Baseline CBF recording was conducted in the previously described manner, and each analysis of CBF was normalized to fold-change over baseline CBF Hz (treatment/baseline).²⁸

HSNEC Viability LDH Assay

HSNEC cell viability was tested using a Lactate Dehydrogenase (LDH) assay as previously described.²⁹ Relative cell viability in the presence of ciprofloxacin and azithromycin is reflective of concentrations of released LDH enzyme originating from damaged HSNECs. Concentration gradients generated from internal standards (with positive controls) provided by the manufacturer render a theoretical kit detection range in biological samples containing IL-8. In this study, the relative cell viability of HSNECs was measured after treatment with ciprofloxacin and azithromycin with concentrations similar to that of drug released from the CASS. Based on the in vitro drug release profile from our previous study, the average dose of ciprofloxacin released daily was 2 μg per day with maximum 2.48±0.46 μg/day at week 3. In regard to azithromycin, an average of approximately 108 μg of azithromycin was released daily for 4 weeks. To reproduce in-vitro drug release conditions where ciprofloxacin/azithromycin remain on the epithelial surface for an entire day, the HSNECs cultured at ALI were treated up to 6 hours with 0.5 μg/ml of ciprofloxacin and/or 30 μg/ml of azithromycin at the apical surface. Released LDH at 3 and 6 hours after incubation is measured with a coupled enzymatic reaction that converts a tetrazolium salt (iodonitrotetrazolium (INT)) into the red color formazan (Cytoscan™ LDH cytotoxicity assay, G-biosciences, St. Louis, Mo.). Quantification of LDH was determined from the concentration of the converted formazan within the 20 minutes of incubation. The resulting formazan absorbs maximally at 492 nm and can be measured quantitatively at 490 nm. Total LDH in each sample was expressed as ng/ml.

Statistical Analysis

All experiments were performed at least in triplicate. Statistical analysis was performed with GraphPad Prism 6.0 (La Jolla, Ca) with significance set at p<0.05. For assessing the anti-inflammatory activity of azithromycin and/or ciprofloxacin, a one-way ANOVA was performed with a Tukey's multiple comparison test for all experiments except the TEER analysis. Normalized values for relative TEER were expressed as ±standard error of the mean.

### Results

Azithromycin Inhibits Production of IL-8

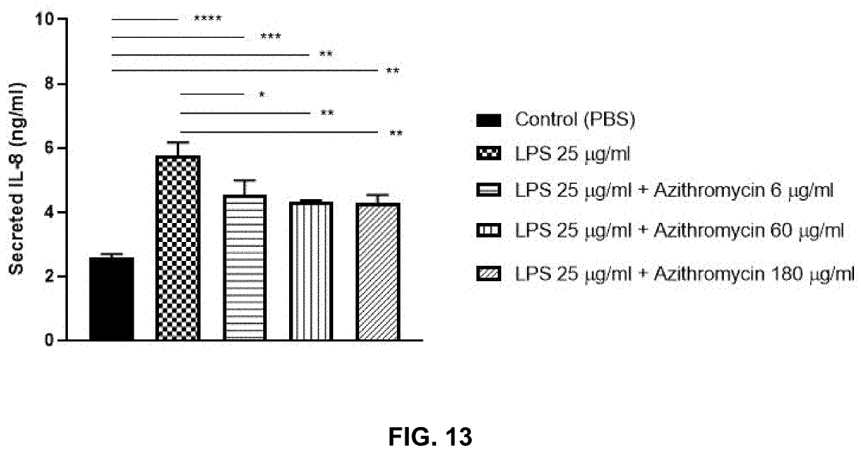

To evaluate the anti-inflammatory effects of azithromycin on HSNEC, cells were stimulated with 25 μg/ml of P. aeruginosa lipopolysaccharide (LPS) for 2 hours, and treated with incremental azithromycin concentrations (6, 60, and 180 μg/ml) (FIG. 13). After a 24-hour incubation period, the basolateral media was harvested and secreted IL-8 was measured. Compared to the PBS treated group (2.656+/−0.150 ng/mL), a significant increase in IL-8 secretion was observed in the P. aeruginosa LPS treated group (5.770+/−0.395 ng/mL, p<0.0001), indicating P. aeruginosa LPS stimulates the production of inflammatory cytokines including IL-8 from HSNECs. When treated with azithromycin (6, 60, and 180 μg/ml), there was a significant reduction in IL-8 production compared to those treated with LPS alone (4.579+/−0.399, 4.312+/−0.057 and 4.269+/−0.258 ng/mL, respectively, p<0.05, n=3 per condition). However, there was no statistically significant dose-dependent response among those cells treated with different concentrations of azithromycin.

Azithromycin-Dependent Reduction of IL-8 in the Presence of Ciprofloxacin

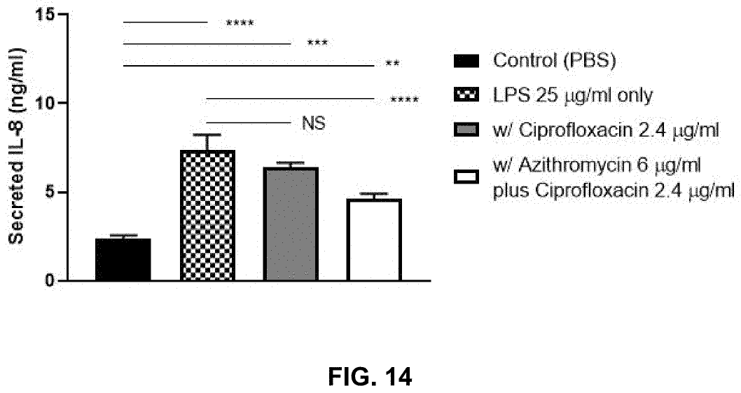

To study the anti-inflammatory effect of azithromycin in the presence of ciprofloxacin, HSNECs were treated with the lowest concentration of azithromycin (6 μg/ml) from our previous dose-response experiment, and co-incubated with ciprofloxacin at 2.4 μg/ml concentration—a concentration similar to the maximal release rate observed in our prior in vitro releasing study (2.48±0.46 μg/day at week 3). As anticipated, secreted IL-8 following P. aeruginosa LPS exposure was significantly increased compared to the PBS control (7.35+/−0.89 ng/ml of IL-8 vs. 2.38+/−0.18 ng/ml of IL-8, p<0.01, n=3) (FIG. 14). Co-treatment with azithromycin and ciprofloxacin significantly reduced the production of IL-8 (4.61+/−0.29 ng/ml, p<0.01, n=3). There was no meaningful reduction in secreted IL-8 in the presence of ciprofloxacin (2.4 μg/ml) alone with P. aeruginosa LPS exposure compared to LPS control (6.40+/−0.26 ng/mL vs. 7.35+/−0.89 ng/ml, p=0.29, n=3). This suggests that the anti-inflammatory activity is solely due to azithromycin.

Azithromycin and Ciprofloxacin do not Affect HSNEC Tight Junction Integrity

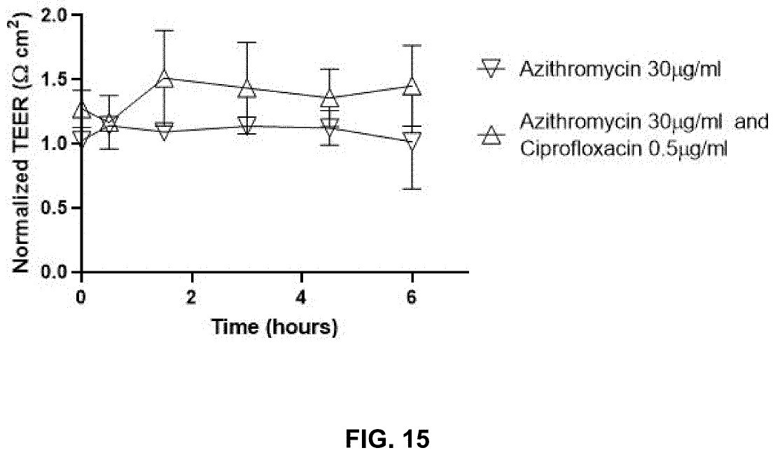

To study the integrity of HSNECs in the presence of azithromycin (30 μg/ml) and/or ciprofloxacin (0.5 μg/ml), the transepithelial electrical resistance (TEER) was assessed (FIG. 15). The TEER was measured at predetermined time points up to 6 hours: 0, 30, 90, 180, 270, and 360 minutes. Each collected TEER values at different time point was normalized against the average values obtained from control groups (PBS). There was no significant reduction in normalized TEER over time. Compared to the azithromycin group (30 μg/ml), the azithromycin/ciprofloxacin (30 μg/ml and 0.5 μg/ml, respectively) group displayed similar or mildly greater resistance compared to control groups (PBS). For example, the normalized TEER values of the 30 μg/ml azithromycin group at 3 hours were 1.14+/−0.06 Ω/cm², whereas the azithromycin/ciprofloxacin group was 1.43+/−0.36 Ω/cm². Similarly, it was found that the azithromycin/ciprofloxacin group displayed a TEER of 1.45+/−0.31 Ω/cm² after 6 hours incubation, which is higher than that of the azithromycin group (1.02+/−0.37 Ω/cm²). There was no significant difference between groups. These findings confirm that the application of azithromycin and ciprofloxacin does not disrupt the integrity of the epithelial membrane, including tight junctions.

Paracellular Permeability of 10 kDa FITC Dextran Particles is Unaffected by Azithromycin and Ciprofloxacin

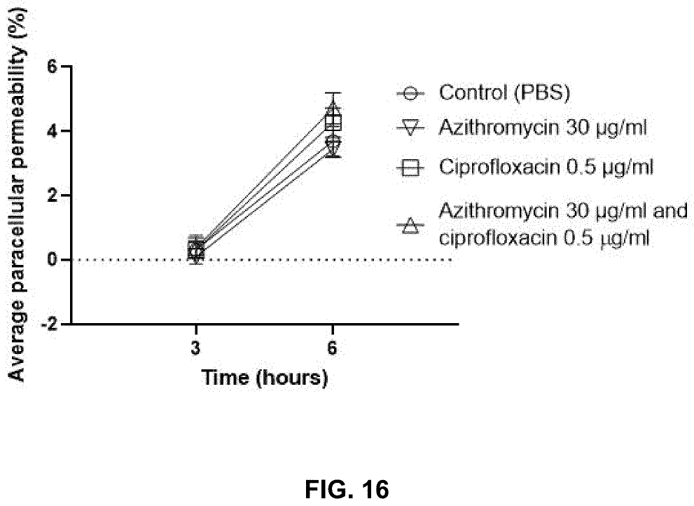

Using 10 kDa FITC dextran particles, the effect of azithromycin and ciprofloxacin on paracellular permeability of HSNECs was evaluated. (FIG. 16). After 3 hours of incubation, average paracellular permeabilities (%) of 60 kDa dextran particles were as follows: control=0.32+/−0.45, azithromycin 30 μg/ml=0.10+/−0.15, ciprofloxacin 0.5 μg/ml=0.31+/−0.26, and azithromycin 30 μg/ml/ciprofloxacin 0.5 μg/ml=0.37+/−0.31 (n=3). Likewise, after 6 hours incubation the results were as follows: control=3.70+/−0.46, azithromycin 30 μg/ml=3.44+/−0.24, ciprofloxacin 0.5 μg/ml=4.27+/−0.45, and azithromycin 30 μg/ml/ciprofloxacin 0.5 μg/ml=4.72+/−0.48 (n=3). As shown in FIG. 4, there is no significant difference between the groups when compared to the control (PBS) group when analyzed at each time point. In each group, low percentages of loaded 10 kDa FITC dextran particles were found on the basolateral surface of the HSNECs at each time point indicating retained integrity with exposure to azithromycin and ciprofloxacin.

Azithromycin and Ciprofloxacin do not Decrease CBF

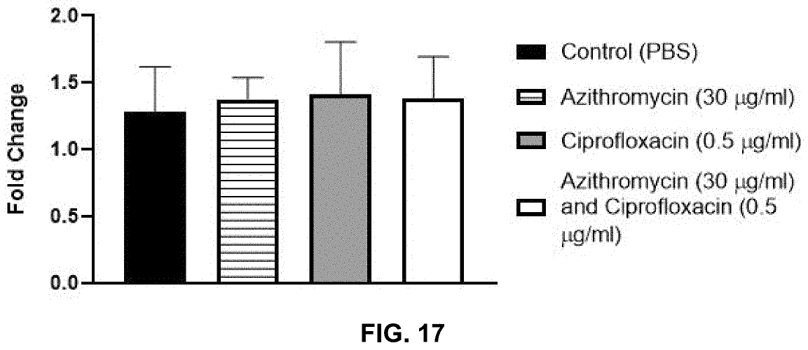

By comparing CBF between groups, changes can reflect diminished function of the mucociliary clearance (MCC) apparatus caused by azithromycin and/or ciprofloxacin. Study drugs (azithromycin (30 μg/ml), ciprofloxacin (0.5 μg/ml), and azithromycin/ciprofloxacin (30 μg/ml and 0.5 μg/ml)) were administered to the apical surface of HSNECs and compared to controls (PBS) (FIG. 17). There was no statistically significant difference in CBF between the groups. The fold changes were as follows: PBS, 1.28+/−0.28 CBF fold changes; azithromycin (30 μg/ml), 1.38+/−0.13 CBF fold changes (p=0.8949); ciprofloxacin (0.5 μg/ml), 1.41+/−0.32 CBF fold changes (p=0.8937); azithromycin/ciprofloxacin (30 μg/ml and 0.5 μg/ml, respectively), 1.39+/−0.25 CBF fold changes (p=0.81).

Ciprofloxacin and Azithromycin are not Toxic to HSNEC

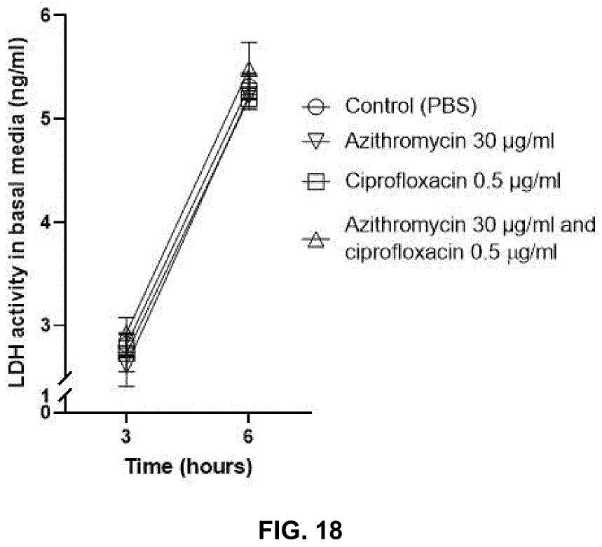

The basolateral media was also collected to measure LDH, which is a cytoplasmic enzyme that leaks extracellularly when the plasma membrane is damaged. As shown in FIG. 18, there was no significant difference of LDH concentrations between groups. Compared to control (PBS) groups, all 3 study groups of HSNECs (azithromycin 30 μg/ml, ciprofloxacin 0.5 μg/ml, and azithromycin 30 μg/ml/ciprofloxacin 0.5 μg/ml) produced similar amounts of LDH at 3 and 6 hours after incubation. After 3 hours, LDH concentrations were as follows: control=2.815+/−0.108, azithromycin 30 μg/ml=2.614+/−0.122, ciprofloxacin 0.5 μg/ml=2.729+/−0.177, and azithromycin 30 μg/ml/ciprofloxacin 0.5 μg/ml=2.930+/−0.147 (n=3 per condition). Similarly, LDH concentrations at 6 hours incubation were as follows: control=5.314+/−0.122, azithromycin 30 μg/ml=5.228+/−0.122, ciprofloxacin 0.5 μg/ml=5.199+/−0.108, and azithromycin 30 μg/ml/ciprofloxacin 0.5 μg/ml=5.486+/−0.254 (n=3 per condition). Azithromycin and ciprofloxacin do not have a detrimental effect on the cellular viability of HSNECs when applied at concentrations attainable from the CASS.

### Discussion

It was demonstrated that azithromycin exhibited significant anti-inflammatory activity at attainable concentrations released from the CASS. P. aeruginosa LPS-stimulated IL-8 secretion was significantly reduced without compromising the integrity or function of HSNECs. The decreased production of IL-8 from HSNECs treated with azithromycin corroborates other published studies showing the anti-inflammatory effect of macrolides including azithromycin in vitro and in vivo.³⁷ Moreover, a recent study identified that sub-inhibitory azithromycin may also reduce S. aureus exoproteins that induce pro-inflammatory cytokines from HSNECs.³⁸ In a study using primary nasal-polyps epithelial cells, several macrolides reduced the production of IL-8 under Escherichia coli serotype 055:85 LPS-stimulation. CRS Patients with nasal polyps (CRSwNP) showed reduced polyp size and lower IL-8 levels in nasal lavage following treatment with macrolide antibiotics. The combination of azithromycin and ciprofloxacin did not affect TEER (maintenance of tight junctions), paracellular permeability (toxicity-induced leaking of substrate), LDH concentrations (measurement of cell toxicity and death), or CBF (cell function) indicating the CASS is highly promising from a safety standpoint as an antibiotic-based intervention for recalcitrant chronic infections in CRS. Advantages of the CASS include extended concurrent delivery of ciprofloxacin and azithromycin to reduce mucosal inflammation, eradication of pre-existing biofilms, and abrogation of new biofilms.

Throughout this publication, various publications are referenced. The disclosures of these publications in their entireties are hereby incorporated by reference into this application in order to more fully describe the methods, compositions, and compounds herein.

Various modification and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

### REFERENCES

1. Schleimer R P. Immunopathogenesis of Chronic Rhinosinusitis and Nasal Polyposis. Annual Review of Pathology: Mechanisms of Disease 2017; 12:331-357.
2. Bendouah Z, Barbeau J, Hamad W A, Desrosiers M. Biofilm formation by Staphylococcus aureus and Pseudomonas aeruginosa is associated with an unfavorable evolution after surgery for chronic sinusitis and nasal polyposis. Otolaryngology—Head and Neck Surgery 2006; 134:991-996.
3. Singhal D, Psaltis A J, Foreman A, Wormald P-J. The Impact of Biofilms on Outcomes after Endoscopic Sinus Surgery. American Journal of Rhinology & Allergy 2010; 24:169-174.
4. Illing E A, Woodworth B A. Management of the upper airway in cystic fibrosis. Curr Opin Pulm Med 2014; 20:623-631.
5. Fastenberg J H, Hsueh W D, Mustafa A, Akbar N A, Abuzeid W M. Biofilms in chronic rhinosinusitis: Pathophysiology and therapeutic strategies. World Journal of Otorhinolaryngology—Head and Neck Surgery 2016; 2:219-229.
6. Cho D Y, Hoffman K, Skinner D et al. Tolerance and pharmacokinetics of a ciprofloxacin-coated sinus stent in a preclinical model. Int Forum Allergy Rhinol 2017; 7:352-358.
7. Cho D Y, Lim D J, Mackey C et al. Preclinical therapeutic efficacy of the ciprofloxacin-eluting sinus stent for Pseudomonas aeruginosa sinusitis. Int Forum Allergy Rhinol 2018; 8:482-489.
8. Cho D Y, Lim D J, Mackey C et al. Ivacaftor, a Cystic Fibrosis Transmembrane Conductance Regulator Potentiator, Enhances Ciprofloxacin Activity Against Pseudomonas aeruginosa. Am J Rhinol Allergy 2019; 33:129-136.
9. Cho D Y, Lim D J, Mackey C et al. In-vitro evaluation of a ciprofloxacin- and ivacaftor-coated sinus stent against Pseudomonas aeruginosa biofilms. Int Forum Allergy Rhinol 2019; 9:486-492.
10. Lim D-J, Skinner D, Mclemore J et al. In-vitro evaluation of a ciprofloxacin and azithromycin sinus stent for Pseudomonas aeruginosa biofilms. International Forum of Allergy & Rhinology 2020; 10:121-127.
11. Aucamp M, Odendaal R, Liebenberg W, Hamman J. Amorphous azithromycin with improved aqueous solubility and intestinal membrane permeability. Drug Dev Ind Pharm 2015; 41:1100-1108.
12. Qiu S, Zhong X. Macrolides: a promising pharmacologic therapy for chronic obstructive pulmonary disease. Ther Adv Respir Dis 2017; 11:147-155.
13. Blasi F, Cazzola M, Tarsia P et al. Azithromycin and lower respiratory tract infections. Expert Opinion on Pharmacotherapy 2005; 6:2335-2351.
14. Yoon B N, Choi N G, Lee H S, Cho K S, Roh H J. Induction of interleukin-8 from nasal epithelial cells during bacterial infection: the role of IL-8 for neutrophil recruitment in chronic rhinosinusitis. Mediators Inflamm 2010; 2010:813610.
15. Cigana C, Nicolis E, Pasetto M, Assael B M, Melotti P. Anti-inflammatory effects of azithromycin in cystic fibrosis airway epithelial cells. Biochemical and Biophysical Research Communications 2006; 350:977-982.
16. Idris S F, Chilvers E R, Haworth C, McKeon D, Condliffe A M. Azithromycin therapy for neutrophilic airways disease: myth or magic? Thorax 2009; 64:186-189.
17. Zhang S, Fortenberry J A, Cohen N A, Sorscher E J, Woodworth B A. Comparison of vectorial ion transport in primary murine airway and human sinonasal air-liquid interface cultures, models for studies of cystic fibrosis, and other airway diseases. Am J Rhinol Allergy 2009; 23:149-152.
18. Bhargave G, Woodworth B A, Xiong G, Wolfe S G, Antunes M B, Cohen N A. Transient receptor potential vanilloid type 4 channel expression in chronic rhinosinusitis. Am J Rhinol 2008; 22:7-12.
19. Woodworth B A, Antunes M B, Bhargave G, Palmer J N, Cohen N A. Murine tracheal and nasal septal epithelium for air-liquid interface cultures: a comparative study. Am J Rhinol 2007; 21:533-537.
20. Woodworth B A, Tamashiro E, Bhargave G, Cohen N A, Palmer J N. An in vitro model of Pseudomonas aeruginosa biofilms on viable airway epithelial cell monolayers. Am J Rhinol 2008; 22:235-238.
21. Antunes M B, Woodworth B A, Bhargave G et al. Murine nasal septa for respiratory epithelial air-liquid interface cultures. Biotechniques 2007; 43:195-196, 198, 200 passim.
22. Conger B T, Zhang S, Skinner D et al. Comparison of cystic fibrosis transmembrane conductance regulator (CFTR) and ciliary beat frequency activation by the CFTR Modulators Genistein, VRT-532, and UCCF-152 in primary sinonasal epithelial cultures. JAMA Otolaryngol Head Neck Surg 2013; 139:822-827.
23. Zhang S, Blount A C, McNicholas C M et al. Resveratrol enhances airway surface liquid depth in sinonasal epithelium by increasing cystic fibrosis transmembrane conductance regulator open probability. PLoS One 2013; 8:e81589.
24. Zhang S, Skinner D, Hicks S B et al. Sinupret activates CFTR and TMEM16A-dependent transepithelial chloride transport and improves indicators of mucociliary clearance. PLoS One 2014; 9:e104090.
25. Woodworth B A. Resveratrol ameliorates abnormalities of fluid and electrolyte secretion in a hypoxia-Induced model of acquired CFTR deficiency. Laryngoscope 2015; 125 Suppl 7:S1-S13.
26. Tipirneni K E, Grayson J W, Zhang S et al. Assessment of acquired mucociliary clearance defects using micro-optical coherence tomography.

Int Forum Allergy Rhinol 2017; 7:920-925.
27. Srinivasan B, Kolli A R, Esch M B, Abaci H E, Shuler M L, Hickman J J. TEER measurement techniques for in vitro barrier model systems. J Lab Autom 2015; 20:107-126.
28. Cho D Y, Skinner D, Zhang S et al. Cystic fibrosis transmembrane conductance regulator activation by the solvent ethanol: implications for topical drug delivery. Int Forum Allergy Rhinol 2016; 6:178-184.
29. Kumar P, Nagarajan A, Uchil P D. Analysis of Cell Viability by the Lactate Dehydrogenase Assay. Cold Spring Harb Protoc 2018; 2018.
30. Barshak M B, Durand M L. The role of infection and antibiotics in chronic rhinosinusitis. Laryngoscope Investigative Otolaryngology 2017; 2:36-42.
31. Fastenberg J H, Hsueh W D, Mustafa A, Akbar N A, Abuzeid W M. Biofilms in chronic rhinosinusitis: Pathophysiology and therapeutic strategies. World J Otorhinolaryngol Head Neck Surg 2016; 2:219-229.
32. Foreman A, Jervis-Bardy J, Wormald P J. Do biofilms contribute to the initiation and recalcitrance of chronic rhinosinusitis? Laryngoscope 2011; 121:1085-1091.
33. Tateda K, Comte R, Pechere J-C, Köhler T, Yamaguchi K, Van Delden C. Azithromycin Inhibits Quorum Sensing in <em>Pseudomonas aeruginosa</em>. Antimicrobial Agents and Chemotherapy 2001; 45:1930-1933.
34. Amali A, Saedi B, Rahavi-Ezabadi S, Ghazavi H, Hassanpoor N. Long-term postoperative azithromycin in patients with chronic rhinosinusitis: A randomized clinical trial. Am J Rhinol Allergy 2015; 29:421-424.
35. Videler W J, Badia L, Harvey R J et al. Lack of efficacy of long-term, low-dose azithromycin in chronic rhinosinusitis: a randomized controlled trial. Allergy 2011; 66:1457-1468.
36. Karma P, Pukander J, Penttila M. Azithromycin concentrations in sinus fluid and mucosa after oral administration. Eur J Clin Microbiol Infect Dis 1991; 10:856-859.
37. Yamada T, Fujieda S, Mori S, Yamamoto H, Saito H. Macrolide Treatment Decreased the Size of Nasal Polyps and IL-8 Levels in Nasal Lavage. American Journal of Rhinology 2000; 14:143-148.
38. Hu H, Ramezanpour M, Hayes A J et al. Sub-Inhibitory Clindamycin and Azithromycin reduce S. aureus Exoprotein Induced Toxicity, Inflammation, Barrier Disruption and Invasion. J Clin Med 2019; 8.
39. Stokes J M, Lopatkin A J, Lobritz M A, Collins J J. Bacterial Metabolism and Antibiotic Efficacy. Cell Metab 2019; 30:251-259.
40. Mustafa M-H, Khandekar S, Tunney M M et al. Acquired resistance to macrolides in <em>Pseudomonas aeruginosa</em> from cystic fibrosis patients. European Respiratory Journal 2017; 49:1601847.
41. Drlica K, Zhao X. Mutant selection window hypothesis updated. Clin Infect Dis 2007; 44:681-688.
42. Drlica K. The mutant selection window and antimicrobial resistance. J

Antimicrob Chemother 2003; 52:11-17.
43. Yeh P J, Hegreness M J, Aiden A P, Kishony R. Drug interactions and the evolution of antibiotic resistance. Nature Reviews Microbiology 2009; 7:460-466.
44. Lister P D, Wolter D J, Hanson N D. Antibacterial-resistant Pseudomonas aeruginosa: clinical impact and complex regulation of chromosomally encoded resistance mechanisms. Clin Microbiol Rev 2009; 22:582-610.
45. Guss J, Abuzeid W M, Doghramji L, Edelstein P H, Chiu A G. Fluoroquinolone-resistant Pseudomonas aeruginosa in chronic rhinosinusitis. ORL J Otorhinolaryngol Relat Spec 2009; 71:263-267.
46. Elies W. [Current therapeutical management, new antibiotics and treatment of Pseudomonas aeruginosa in bacterial ENT-infections]. Laryngorhinootologie 2002; 81:40-45.
47. Lim D J, McCormick J, Skinner D et al. Controlled delivery of ciprofloxacin and ivacaftor via sinus stent in a preclinical model of Pseudomonas sinusitis. Int Forum Allergy Rhinol 2020; 10:481-488.

1. A substrate comprising a first surface, wherein a first layer comprising a first bioactive agent is adjacent to the first surface of the substrate, and a second layer comprising a second bioactive agent is adjacent to the first layer, wherein the second bioactive agent is more hydrophobic than the first bioactive agent.

2. The substrate of claim 1, wherein the first bioactive agent comprises an antibiotic.

3. The substrate of claim 1, wherein the first bioactive agent comprises ciprofloxacin, levofloxacin, norfloxacin, ofloxacin, perfloxacin, lomefloxacin, fleroxacin, sparfloxacin, grepafloxacin, trovafloxacin, clinafloxacin, gemifloxacin, enoxacin, sitafloxacin, nadifloxacin, tosulfloxacin, cinnoxacin, rosoxacin, miloxacin, moxifloxacin, gatifloxacin, nalidixic acid, nadifloxacin, oxolinic acid, pefloxacin, pirimidic acid, pipernidic acid, rufloxacin, temafloxacin, trovafloxacin, besifloxacin, or any combination thereof.

4. (canceled)

5. The substrate of claim 1, wherein the first bioactive agent comprises a plurality of nanoparticles, wherein the nanoparticles comprise the first antibiotic.

6. (canceled)

7. The substrate of claim 5, wherein the nanoparticles comprise a polylactide, a polyglycolide, a polylactide-co-glycolide, a polyesteramide, a polyorthoester, a poly-p-hydroxybutyric acid, a polyanhydride, a polydiene, a polyalkylene glycol, a polymethacrylate, a polyvinyl ether, a polyvinyl alcohol, a polyvinyl chloride, a polyvinyl ester, a polycarbonate, a polyester, a cellulose ether, a cellulose ester, a polysaccharide, a polycaprolactone, starch, or any combination thereof.

8. (canceled)

9. The substrate of claim 1, wherein the first layer comprises a plurality of nanoparticles, wherein the nanoparticles particles are homogeneously dispersed in a first polymer.

10. (canceled)

11. The substrate of claim 9, wherein the first polymer comprises a homopolymer or a copolymer of an acrylate, a methacrylate, an acrylamide, a methacrylamide, acrylic acid, methacrylic acid, an acrylic acid ester, a methacrylic acid ester, or any combination thereof.

12. The substrate of claim 1, wherein the first layer has a thickness of from about 50 pm to about 200 pm.

13. The substrate of claim 1, wherein the first bioactive agent is an antibiotic present in the first layer in the amount of about 1 pg to 500 mg.

14-16. (canceled)

17. The substrate of claim 1, wherein the second bioactive agent comprises a second antibiotic.

18. The substrate of claim 17, wherein the second antibiotic is present in the second layer in the amount of about 5 pg to 500 mg.

19-22. (canceled)

23. The substrate of claim 1, wherein the second layer comprises a plurality of nanoparticles, wherein the nanoparticles comprise the second bioactive agent.

24. (canceled)

25. The substrate of claim 23, wherein the nanoparticles comprise a polylactide, a polyglycolide, a polylactide-co-glycolide, a polyesteramide, a polyorthoester, a poly-p-hydroxybutyric acid, a polyanhydride, a polydiene, a polyalkylene glycol, a polymethacrylate, a polyvinyl ether, a polyvinyl alcohol, a polyvinyl chloride, a polyvinyl ester, a polycarbonate, a polyester, a cellulose ether, a cellulose ester, a polysaccharide, a polycaprolactone, starch, or any combination thereof.

26. (canceled)

27. The substrate of claim 1, wherein the second layer comprises a plurality of nanoparticles, wherein the nanoparticles particles are homogeneously dispersed in a second polymer.

28. (canceled)

29. The substrate of claim 27, wherein the second polymer comprises a homopolymer or a copolymer of an acrylate, a methacrylate, an acrylamide, a methacrylamide, acrylic acid, methacrylic acid, an acrylic acid ester, a methacrylic acid ester, or any combination thereof.

30. The substrate of claim 1, wherein the second layer has a thickness of from about 50 pm to about 500 pm.

31-36. (canceled)

37. A method for treating or preventing a bacterial infection in a subject comprising administering to the subject the substrate of claim 1.

38. A method for treating or preventing inflammation in a subject comprising administering to the subject the substrate of claim 1.

39-41. (canceled)

42. The method of claim 37, wherein the subject has chronic inflammatory disease in the upper airway system

43. The method of claim 37, wherein the subject has chronic rhinosinusitis, hyposmia/anosmia, chronic rhinitis, allergic rhinitis, or vasomotor rhinitis.

44-47. (canceled)
